(19) United States
(12) Patent Application Publication
(10) Pub. No.: US 2008/0119406 A1
(43) Pub. Date: May 22, 2008

(54) Osteoblast Growth Factor

(76) Inventors: Tuan Phan, Success (AU); Jiake Xu, Karrinyup (AU); Ming Hao Zheng, City Beach (AU)

Correspondence Address: DUANE MORRIS, LLP;IP DEPARTMENT, 30 SOUTH 17TH STREET, PHILADELPHIA, PA, 19103-4196, US

(21) Appl. No.: 11/587,218

(22) PCT Filed: Apr. 13, 2005
(86) PCT No.: PCT/AU05/00526

§ 371 Date: Aug. 13, 2007

(30) Foreign Application Priority Data
Apr. 19, 2004 (AU) 2004902048

(51) Int. Cl. A61K 38/00; A61P 19/00

(52) U.S. Cl. 514/12; 514/13; 514/14; 514/15

(57) ABSTRACT

The present invention provides to the isolation of a small cysteine rich secretory protein from a haematopoietic macrophage cell line, which is capable of elevating cytosolic calcium levels. In particular, the present invention provides a method of treating or preventing bone disorders or diseases comprising the step of administering a composition comprising caltrin or functionally active fragment thereof to an individual in a need thereof.

### FIELD OF THE INVENTION

The present invention relates to the isolation of a small cysteine rich secretory protein from a haematopoietic macrophage cell line, which is capable of elevating cytosolic calcium levels. In particular, the present invention relates to an isolated protein called caltrin and its use in the treatment or prevention of bone disorders and diseases.

### BACKGROUND OF THE INVENTION

Bone is a crucial tissue that provides internal skeletal support for every organ as well as forming and structuring the entire human frame. In addition, bone is the home for the formation of haematopoietic cells and the regulation of blood calcium. Due to its importance in the human body, bone needs to be continuously replenished in order to maintain its strength and structural integrity. This replenishment, also known as bone remodelling, is controlled by two equal, but opposing, forces: bone formation by osteoblasts and bone destruction or resorption by osteoclasts. Intimate communication between these cells is an integral element in maintaining bone homeostasis. Despite the vigorous regulation and control of bone equilibrium, changes in remodelling can occur, either by defects in the cell or obstruction of the intercellular communication between the cells, which leads to debilitating bone diseases such as osteoporosis, osteopetrosis, osteogenesis imperfecta and even Paget's Disease. The treatment of these diseases can cost billions of dollars a year, and diseases such as osteoporosis have a relatively high incidence rate.

To date, no completely effective treatment for, or prevention of, bone disorders or diseases has been proposed. For example, osteopenia treatments have been based on inhibiting further bone resorption, eg., by 1) inhibiting the differentiation of haematopoietic mononuclear cells into mature osteoclasts, 2) by directly preventing osteoclast-mediated bone resorption, or 3) by affecting the hormonal control of bone resorption. Drug regimens used for the treatment of osteoporosis have included calcium supplements, oestrogen, calcitonin and diphosphonates. Vitamin D₃ and its metabolites, known to enhance calcium and phosphate absorption have also been trialed. However, none of these therapies have been able to stimulate regeneration of new bone tissue. In addition, all of the agents used have only a transient effect on bone remodelling. Consequently, while in some cases the progression of a bone disorder or disease may be halted or slowed, patients with significant bone deterioration remain at risk. This is particularly prevalent in disorders such as osteoporosis where early diagnosis is difficult and/or rare and significant structural deterioration of the bone already may have occurred.

Thus, there is a continuing need to develop methods of replacing bone that result in bone that is substantially similar, structurally and architecturally, to the type of bone lost.

### SUMMARY OF THE INVENTION

The inventors have now discovered and described a novel nucleic acid molecule encoding a small cysteine rich secretory protein isolated from a haematopoietic macrophage cell line, which is capable of increasing proliferation of osteoblasts. This protein has been called calcium transport inhibitor, or caltrin.

Accordingly, in a first aspect the present invention provides an isolated nucleic acid comprising a nucleotide sequence encoding a peptide having an activity of caltrin, wherein said nucleotide sequence comprises the nucleotide sequence shown in FIG. 1 (SEQ ID NO: 1) or FIG. 2 (SEQ ID NO: 2) or a functionally active fragment thereof.

In one embodiment, the isolated nucleic acid consists essentially of either the nucleotide sequence shown in FIG. 1 (SEQ ID NO: 1) or FIG. 2 (SEQ ID NO: 2).

It will be appreciated by those skilled in the art that the isolated nucleic acid of the present invention may be cDNA, genomic DNA, RNA, or a hybrid molecule thereof. Preferably, the nucleic acid is cDNA having either the nucleotide sequence shown in FIG. 1 (SEQ ID NO: 1) or FIG. 2 (SEQ ID NO: 2).

Also contemplated are isolated nucleic acids, which hybridise under high stringency conditions to a nucleic acid having either the nucleotide sequence shown in FIG. 1 (SEQ ID NO: 1) or FIG. 2 (SEQ ID NO: 2).

The invention also relates to a peptide encoded by either the nucleotide sequence shown in SEQ ID NO: 1 or SEQ ID NO: 2 or a portion thereof. Preferably, the nucleic acid encodes a peptide which has the amino acid sequence shown in FIG. 3 (SEQ ID NO: 3) or FIG. 4 (SEQ ID NO: 4) and having at least one biological activity of caltrin.

Accordingly, in a second aspect the present invention provides an isolated peptide of caltrin, coded for by the nucleotide sequence shown in FIG. 1 (SEQ ID NO: 1) or FIG. 2 (SEQ ID NO: 2) or functionally active fragment thereof.

Preferably, the isolated peptide is about 10 to about 20 amino acids in length. More preferably, about 10 to about 60 amino acids in length. Even more preferably, about 76 amino acids in length. Most preferably, the isolated peptide of caltrin has an amino acid sequence shown in FIG. 3 (SEQ ID NO: 3) or FIG. 4 (SEQ ID NO: 4), or cleaved product thereof.

In a further embodiment, the isolated peptide of caltrin has an amino acid sequence consisting essentially of the amino acid sequence shown in FIG. 3 (SEQ ID NO: 3) or FIG. 4 (SEQ ID NO: 4).

Peptides having an activity of caltrin and having at least 50% identity with a sequence shown in FIG. 3 (SEQ ID NO: 3) are also featured. Peptides having a caltrin activity produced by recombinant expression of a nucleic acid of the invention, and peptides having a caltrin activity prepared by chemical synthesis are also featured by this invention.

In a third aspect the present invention provides a method of producing a caltrin peptide or functionally active fragment thereof comprising the steps of:

i) transforming a host cell with DNA having a nucleotide sequence shown in FIG. 1 (SEQ ID NO: 1) or FIG. 2 (SEQ ID NO: 2) or functionally active fragment thereof, which DNA codes for caltrin; and

ii) isolating and purifying caltrin from host cell.

Preferred peptides have the ability to elevate cytosolic calcium levels in osteoblasts and osteoblast-like cells. Other preferred peptides also have the ability to induce bone formation, which may include stimulation of osteoblasts or proliferation of osteoblasts. Other preferred peptides, either apart from or in addition to the ability to elevate cytosolic calcium levels, have the ability to bind acrogranin (an acidic cysteine-rich glycoprotein of 67 kDa) and/or EMILIN. Such peptides are useful in treating or preventing bone disorders or diseases in a subject.

In a fourth aspect, the present invention provides an antibody specifically reactive with a peptide having caltrin activity.

In a fifth aspect the present invention provides a method of identifying a binding partner ligand which can bind an isolated peptide of caltrin, coded for by the nucleotide sequence shown in FIG. 1 (SEQ ID NO: 1) or FIG. 2 (SEQ ID NO: 2) or functionally active fragment thereof comprising the step of screening a combinatorial library comprising a plurality of member ligands for the ability to bind said isolated peptide of caltrin or functionally active fragment thereof.

In a sixth aspect the present invention provides a method of screening for a modulator of caltrin expression comprising the steps of:

(a) providing a cell expressing caltrin or functionally active fragment thereof;

(b) contacting said cell with a candidate modulator; measuring caltrin expression; and

(c) comparing said caltrin expression in the presence of said candidate modulator with the expression of caltrin in the absence of said candidate modulator; wherein a difference in the expression of caltrin in the presence of said candidate modulator, as compared with the expression of caltrin in the absence of said candidate modulator, identifies said candidate modulator as a modulator of caltrin expression.

It is envisioned that these modulators may be used to enhance or inhibit caltrin expression. Modulators that enhance caltrin expression may be used to promote more efficient bone healing or repair. Yet further, modulators that inhibit caltrin expression may be used to inhibit caltrin function.

In one embodiment, the method of screening provides molecules, which can mediate the biological activity of caltrin via its binding to its receptor. Thus, in a seventh aspect the present invention provides a method of identifying a molecule which can mediate the biological activity of caltrin comprising:

(a) screening a first combinatorial library comprising a plurality of first member ligands for binding to caltrin, thereby identifying one or more receptor-binding ligands;

(b) screening a second library comprising a plurality of second member ligands for the ability to modulate the binding of one or more of said target-binding ligands to said caltrin, thereby obtaining one or more modulators, and

(c) determining which of the modulators can mediate a biological activity of caltrin, said modulator thereby being identified as an activity-mediating ligand.

A peptide having an activity of caltrin can also be used in compositions suitable for pharmaceutical administration. For example, such compositions can be used to treat or prevent bone disorders or diseases in a subject. Accordingly, in an eighth aspect, the present invention provides a composition comprising a therapeutically- or prophylactically-effective amount of caltrin or functionally active peptide fragment thereof together with a pharmaceutically acceptable carrier, wherein said caltrin is coded for by a nucleotide sequence comprising the nucleotide sequence shown in FIG. 1 (SEQ ID NO: 1) or FIG. 2 (SEQ ID NO: 2).

In a ninth aspect, the present invention provides a method of treating or preventing bone disorders or diseases comprising the step of administering a composition comprising caltrin or functionally active fragment thereof to an individual in need thereof.

In a tenth aspect, the present invention provides a method for inducing bone formation comprising the step of administering a composition comprising caltrin or functionally active fragment thereof to an individual in need thereof.

In an eleventh aspect, the present invention provides a method for forming bone matrix comprising the step of administering a composition comprising caltrin or functionally active fragment thereof to an individual in need thereof.

In an twelfth aspect, the present invention provides a method for increasing proliferation of osteoblasts comprising the step of administering a composition comprising caltrin or functionally active fragment thereof to an individual in need thereof.

The lytic bone disease or disorder can be any condition, which results in excessive osteoclastic bone resorption and/or hypercalcemic serum effects. Preferably, the bone disease or disorder is selected from the group consisting of osteoporosis (including post menopausal osteoporosis, male and female senile osteoporosis and corticosteroid induced osteoporosis), osteoarthritis, Paget's disease, osteomalacia, multiple myeloma and other forms of cancer, prolonged bed rest, chronic disuse of a limb, anorexia, microgravity, exogenous and endogenous gonadal insufficiency, bone fracture, non-union, defect, prosthesis implantation, malignancy-related bone loss, and the like.

The individual may be any animal in which excessive osteoclastic bone resorption and/or hypercalcemic serum effects are observed. Consequently, the individual may be any vertebrate animal, including mammals. Suitable mammalian individuals include members of the Orders Primates, Rodentia, Lagomorpha, Cetacea, Carnivora, Perissodactyla and Artiodactyla. Preferably, the animal is a human subject.

In a thirteenth aspect, the present invention provides a method of treating osteopenia, comprising administering systemically to a mammal a composition consisting essentially of caltrin and a pharmaceutically-acceptable carrier, wherein said mammal suffers from osteopenia, and wherein said caltrin comprises an amino acid sequence selected from the group consisting of a sequence:

(a) having at least 70% homology with the residues 1-99 of SEQ ID NO: 3;

(b) having greater than 60% amino acid sequence identity with said SEQ ID NO: 3; and

wherein said caltrin induces bone formation in an in vivo bone assay.

In a fourteenth aspect, the present invention provides a method for restoring loss of bone mass in a mammal afflicted with osteopenia, comprising administering systemically to said mammal a composition consisting essentially of a caltrin and a pharmaceutically-acceptable carrier, wherein said caltrin comprises an amino acid sequence selected from the group consisting of a sequence:

a) having at least 70% homology with the residues 1-99 of SEQ ID NO: 3;

(b) having greater than 60% amino acid sequence identity with said SEQ ID NO: 3; and

wherein said caltrin induces bone formation in an in vivo bone assay.

In a fifteenth aspect, the present invention provides a method for preventing loss of bone mass in a mammal at risk of osteopenia, comprising administering systemically to said mammal a composition consisting essentially of a caltrin and a pharmaceutically-acceptable carrier, wherein said caltrin comprises an amino acid sequence selected from the group consisting of a sequence:

a) having at least 70% homology with the residues 1-99 of SEQ ID NO: 3;

(b) having greater than 60% amino acid sequence identity with said SEQ ID NO: 3; and

wherein said caltrin induces bone formation in an in vivo bone assay.

### BRIEF DESCRIPTION OF THE FIGURES

FIG. 1 shows nucleotide sequence of mouse caltrin (SEQ ID NO: 1).

FIG. 2 shows nucleotide sequence of human caltrin (SEQ ID NO: 2).

FIG. 3 shows deduced amino acid sequence of mouse caltrin (SEQ ID NO: 3).

FIG. 4 shows deduced amino acid sequence of human caltrin (SEQ ID NO: 4).

FIG. 5 shows the identification and preliminary bioinformatic characterisation of caltrin.

FIG. 6(A) shows the expression of caltrin mRNA during osteoclastogenesis, while FIG. 6(B) shows the expression of caltrin mRNA in osteoblasts. FIG. 6(C) shows the tissue distribution of caltrin mRNA.

FIG. 7 shows a Western Blot analysis of caltrin and mock-infected caltrin cell supernatant. Crude supernatant from caltrin-infected (lane 1) and mock-infected (lane 2) are shown.

FIG. 8 shows a fluorescent-based binding profile for recombinant caltrin.

FIG. 9 shows the labelling curve of ¹²⁵I-caltrin.

FIG. 10 shows the qualitative ¹²⁵I Binding Analysis.

FIG. 11 shows the quantitative Binding analysis between ¹²⁵I caltrin and primary calvarial osteoblasts.

FIG. 12 shows a quantitative Scatchard Plot and Dissociation Curve.

FIG. 13 shows that caltrin stimulates osteoblastic proliferation in vitro.

FIG. 14 shows that caltrin stimulates human MG-63 osteoblastic proliferation in vitro.

FIG. 15 shows that caltrin does not stimulate proliferation of RAW_{246.7} cells.

FIG. 16 shows a qualitative osteoblast mineralisation of calcium nodules.

FIG. 17 shows a quantitative osteoblast mineralisation of calcium nodules.

FIG. 18 shows the effect of caltrin on osteoclastogenesis.

FIG. 19 shows the effect of caltrin on osteoclast survival.

FIG. 20 shows the quantitative assessment of the effect of caltrin stimulation on osteoclastic bone resorption.

FIG. 21 shows the alteration in [Ca²⁺]ᵢ after stimulation with 50 ng/mL of caltrin.

FIG. 22 shows the alteration in [Ca²⁺]ᵢ after stimulation with 500 ng/mL of caltrin.

FIG. 23 shows the cytosolic calcium alterations in osteoclasts after stimulation with 100 ng/mL of caltrin.

FIG. 24 shows that caltrin elevates [Ca²⁺]ᵢ by depleting internal calcium store.

FIG. 25 shows the phosphorylation of PI3K-Akt pathway.

FIG. 26 shows the activation of the ERK pathway.

### ABBREVIATIONS

### BEVS Baculovirus Expression Vector System

### BMP Bone Morphogenic Protein

### Caltrin Calcium Transport Inhibitor

### CAII Carbonic Anhydrase II

### [Ca²⁺]_{I} cytosolic calcium concentration

### EMILIN Elastin Microfibril Interface Located Protein

### EGF Epidermal Growth factor.

### ERK Extracellular signal Regulated Kinase

### FACS Fluorescence Activated Cell Sorting

### GPI Glycosylphosphatidylinositol

### IMAC Immobilised Metal Affinity Chromatography

### IP3 Inositolphosphate-3

### IGF Insulin-like Growth Factor

### Ly-6 Lymphocyte Antigen 6

### M-CSF Macrophage Colony Stimulating Factor

### MMPs Matrix Metalloproteinases

### ODOF Osteoclast-Derived Osteoblastic Factor

### OPG Osteoprotegerin

### OPGL Osteoprotegerin Ligand

### PTH Parathyroid Hormone

### PI3K Phosphoinositide-3-kinase

### PLC Phospholipase C

### PDGF Platelet Derived Growth Factor

### RANK Receptor Activator of NFkB

### RANKL Receptor Activator of NFkB ligand

### SVS VII Seminal Vesicle Secretion VII

### TE Tris EDTA

### TRAP Tartrate Resistant Acid Phosphatase

### TGFα,β Transforming Growth Factor Alpha, Beta

### TNFα,β Tumour Necrosis Factor Alpha, Beta

### DETAILED DESCRIPTION OF THE INVENTION

Before describing the present invention in detail, it is to be understood that this invention is not limited to particularly exemplified methods and may, of course, vary. It is also to be understood that the terminology used herein is for the purpose of describing particular embodiments of the invention only, and is not intended to be limiting which will be limited only by the appended claims.

All publications, patents and patent applications cited herein, whether supra or infra, are hereby incorporated by reference in their entirety. However, publications mentioned herein are cited for the purpose of describing and disclosing the protocols, reagents and vectors which are reported in the publications and which might be used in connection with the invention. Nothing herein is to be construed as an admission that the invention is not entitled to antedate such disclosure by virtue of prior invention.

Furthermore, the practice of the present invention employs, unless otherwise indicated, conventional immunological and molecular biological techniques and pharmacology within the skill of the art. Such techniques are well known to the skilled worker, and are explained fully in the literature. See, eg., Coligan, Dunn, Ploegh, Speicher and Wingfield “Current protocols in Protein Science” (1999) Volume I and II (John Wiley & Sons Inc.); Sambrook et al., (Molecular Cloning: A Laboratory Manual, 2nd Edition, Cold Spring Harbor Laboratory press (1989); and Bailey, J. E. and Ollis, D. F., Biochemical Engineering Fundamentals, McGraw-Hill Book Company, NY, 1986.

It must be noted that as used herein and in the appended claims, the singular forms “a,” “an,” and “the” include plural reference unless the context clearly dictates otherwise. Thus, for example, a reference to “a nucleic acid” includes a plurality of such nucleic acids, and a reference to “an isolated peptide” is a reference to one or more peptides, and so forth. Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

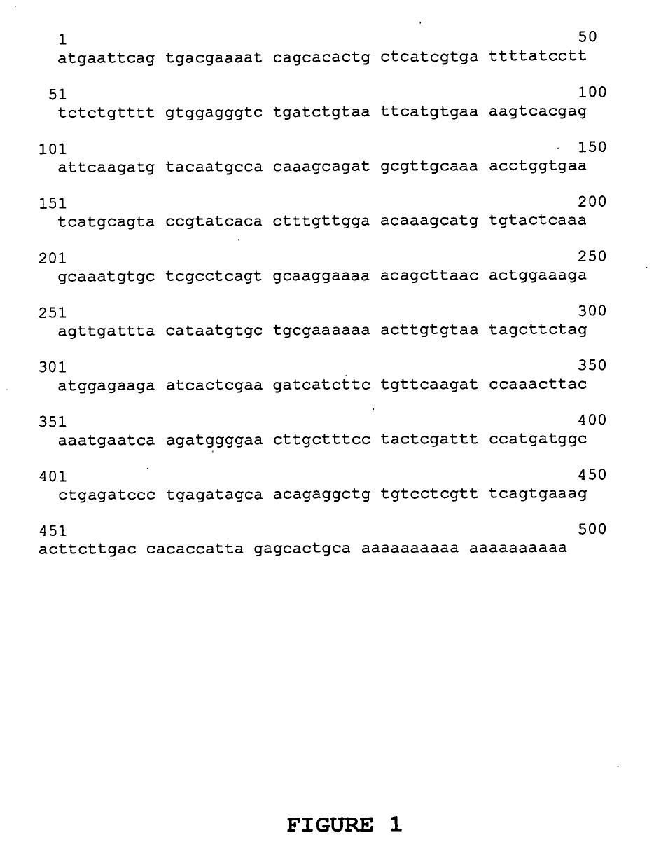

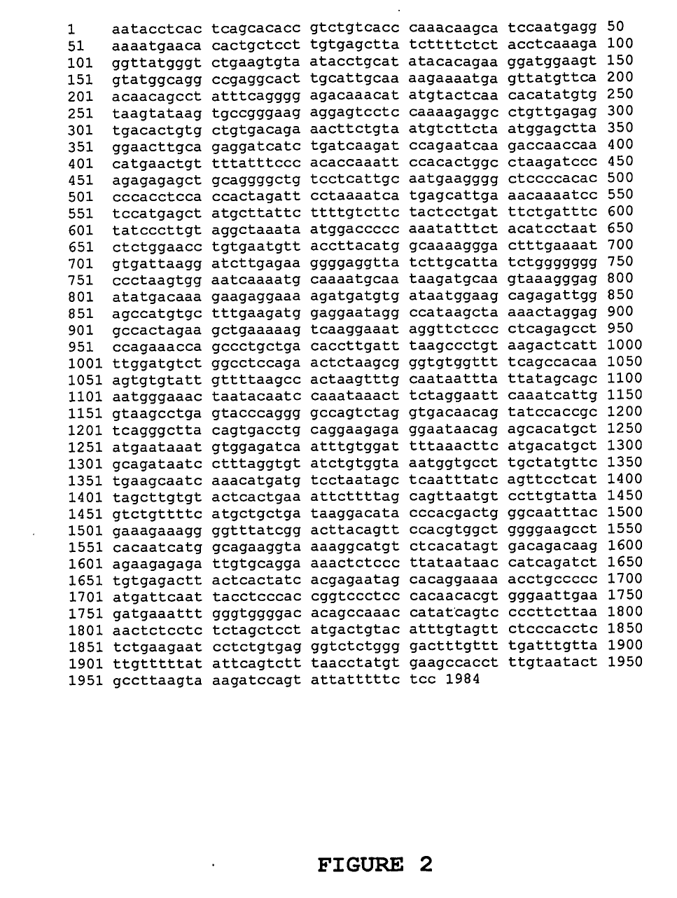

This invention relates to an isolated nucleic acid encoding a peptide having at least one biological activity of caltrin. The term “nucleic acid” as used herein refers to deoxyribonucleic acid (DNA) and ribonucleic acid (RNA) in all their forms, ie., single and double-stranded DNA, cDNA, genomic DNA, mRNA, and the like. In one embodiment, the nucleic acid is a cDNA comprising either the nucleotide sequence shown in FIG. 1 (SEQ ID NO: 1) or FIG. 2 (SEQ ID NO: 2) or a functionally active fragment thereof.

In one embodiment, the nucleic acid is a cDNA comprising the nucleotide sequence shown in FIG. 1 (SEQ ID NO: 1) or FIG. 2 (SEQ ID NO: 2) or a functionally active fragment thereof.

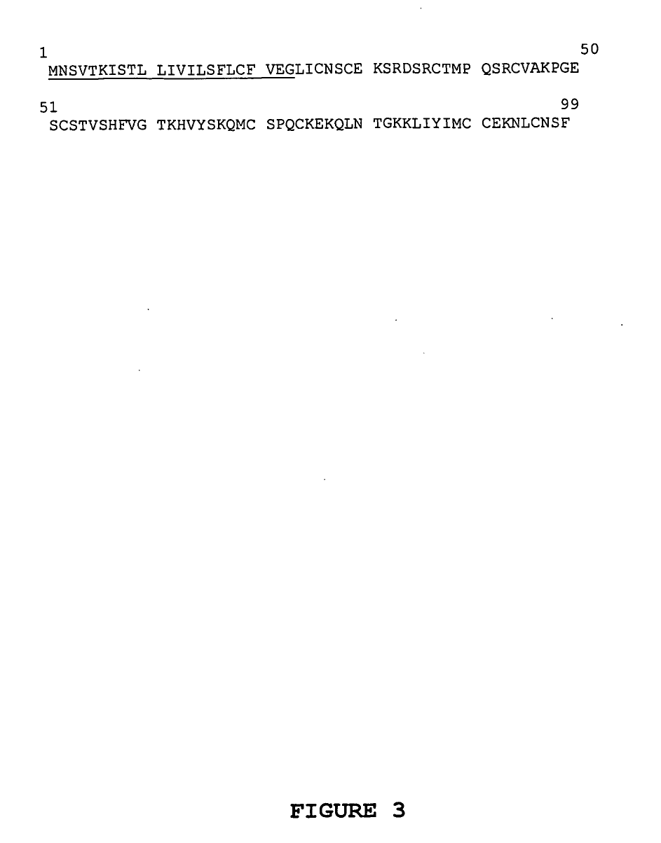

The cDNA shown in FIG. 1 (SEQ ID NO: 1) encodes a caltrin protein, which is predicted to include a 23 amino acid signal peptide encoded by base 1 through base 69. This signal peptide is not found in the mature caltrin protein, which is encoded by bases 70 through 228. The deduced amino acid sequence of caltrin based on this cDNA is shown in FIG. 3 (SEQ ID NO: 3). The cDNA encodes a mature peptide having a predicted molecular weight of 8,500 Da (8.5 kDa), with a pI of 9.0.

In one embodiment of this invention there is provided an isolated nucleic acid molecule comprising nucleotide sequences encoding caltrin or functionally active fragment thereof, which encodes a peptide having at least one biological activity of caltrin, and/or equivalents of such nucleic acids. It will be clearly understood that the term “nucleic acid” also encompasses a full-length molecule encoding a peptide including the caltrin peptides, as well as truncated molecules or altered molecules that code for functionally active derivatives, analogs, homologs or variants thereof.

The term “functionally active,” when used in reference to the caltrin nucleic acid of the present invention, refers to the paradigm in which an alteration to a nucleotide sequence does not necessarily affect the sequences ability to code for a peptide capable of performing substantially the same function as the unaltered “parent” peptide. For example, a nucleotide sequence may be truncated, elongated, or mutated in such a way that the peptide coded by the nucleotide sequence differs from the “parent” sequence, but still codes for a peptide that is capable of functioning in a substantially similar way to the “parent” molecule. Consequently, a functionally active derivative, analog, homolog or variant of caltrin of the present invention will have a nucleotide sequence which differs from the nucleotide sequence shown in FIG. 1 or FIG. 2, but the peptide coded for by the functionally active derivative, analog, homolog or variant is capable of displaying one or more known functional activities associated with caltrin. Such modifications may be deliberate, as by site-directed mutagenesis, or may be spontaneous. All of the peptides produced by these modifications are included herein as long as the caltrin activity is present.

It will be appreciated by those skilled in the art that a functionally active derivative, analog, homolog or variant of caltrin of the present invention can vary substantially outside regions of importance eg receptor binding sites; however, regions of high sequence conservation between caltrin isolated from different mammalian species (see, for example, Table 1) are likely to code for important regions such as receptor binding sites and the like. Accordingly, it is likely that mutations in these highly conserved regions will not generate functionally active derivatives, analogs, homologs or variants.

Equivalent nucleotide sequences will include sequences that differ by one or more nucleotide substitutions, additions or deletions, such as allelic variants, and will also include sequences that differ from the nucleotide sequence encoding caltrin shown in FIG. 1 (SEQ ID NO: 1) or FIG. 2 (SEQ ID NO: 2) due to the degeneracy of the genetic code. Equivalents will also include nucleotide sequences that are “substantially homologous” ie at least about 85%, preferably at least about 90%, and most preferably at least about 95%, of the nucleotides match over the defined length of the nucleotide sequences. Sequences that are substantially similar can be identified in a Southern hybridisation experiment, for example under high, medium or low stringency conditions as defined for that particular system. Defining appropriate hybridisation conditions is within the skill of the art. See eg., Sambrook et al., supra. However, ordinarily, “stringent conditions” for hybridisation or annealing of nucleic acid molecules are those that

(1) employ low ionic strength and high temperature for washing, for example, 0.015M NaCl/0.0015M sodium citrate/0.1% sodium dodecyl sulfate (SDS) at 50° C., or

(2) employ during hybridisation a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C.

An example of medium stringency conditions for hybridisation is the use of 50% formamide, 5×SSC (0.75M NaCl, 0.075M sodium citrate), 50 mM sodium phosphate (pH 6.8), 0.1% sodium pyrophosphate, 5×Denhardt's solution, sonicated salmon sperm DNA (50 μg/mL), 0.1% SDS, and 10% dextran sulfate at 42° C., with washes at 42° C. in 0.2×SSC and 0.1% SDS.

TABLE 1

COMPARISON OF AMINO ACID SEQUENCES OF DIFFERENT
MAMMALIAN CALTRIN MOLECULES

| | |
|---|---|
| Mouse | MNSVTKISTL LIVILSFLCF VEG---LICN SCEKSRDSRC TMPQSRCVAK |
| Rat | ***M****I* ***A****** T*ANTN**** T*NR*EN*E* KNGTGQ*T*P |
| Human | ---MR*MN** *L*S****YL K**---*K** T*IYTEGWK* MAGRGT*I** |
| Mouse | PGESCSTVSH FVGTKHVYSK QMCSPQCKEK QLNTGKKLIY IMCCEKNLCN |
| Rat | E*G****I*I YH*QR**L** ***LGH*E** PHYN*DFM** V***S***** |
| Human | EN*L***TAY *S*D**M**T H**KYK*R*E ESSKRGL*RV TL**DR*F** |
| Mouse | SF |
| Rat | ** |
| Human | V* |

By way of further example, and not intended as limiting, low stringency conditions include those described by Shilo and Weinberg in 1981 (Proc. Natl. Acad. Sci. USA 78:6789-6792). When filters containing DNA are treated using these conditions they are usually pre-treated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 g/ml denatured salmon sperm DNA. Hybridisations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridisation mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and re-exposed to film. Other conditions of low stringency, which may be used are well known in the art (eg., as employed for cross-species hybridisations).

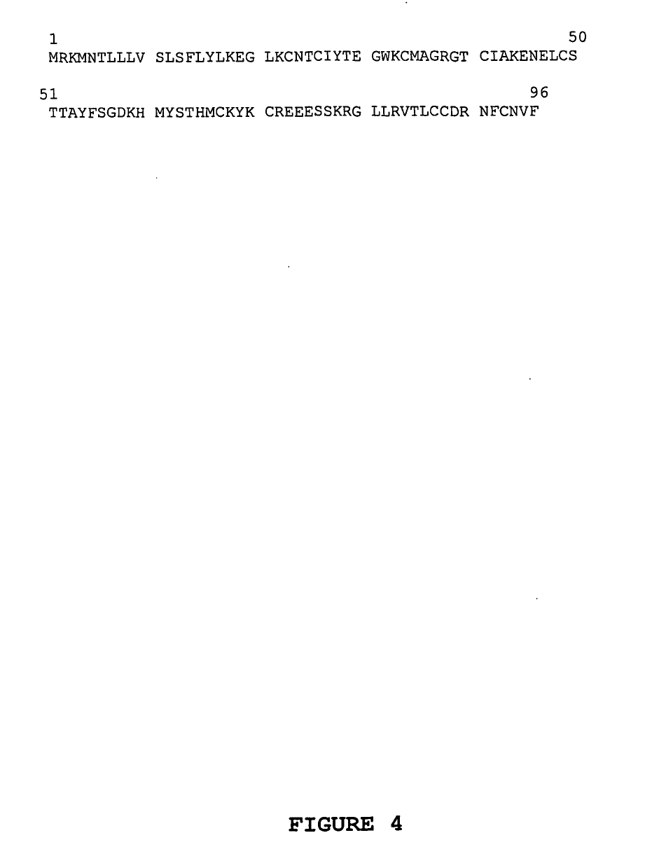

Peptides referred to herein as having caltrin activity are defined herein as peptides that have an amino acid sequence corresponding to all or a portion of the amino acid sequence of caltrin shown in FIG. 3 (SEQ ID NO: 3) or FIG. 4 (SEQ ID NO: 4), which peptide has at least one biological activity of caltrin. For example, a peptide having an activity of caltrin may have the ability to elevate, cytosolic calcium levels in osteoblasts and osteoblast-like cells. Alternatively, or additionally, a peptide having an activity of caltrin may have the ability to induce bone formation, which may include stimulation of osteoblasts or proliferation of osteoblasts. Other preferred peptides, either apart from or in addition to the ability to elevate cytosolic calcium levels, may have the ability to bind acrogranin (an acidic cysteine-rich glycoprotein of 67 kDa) and/or EMILIN.

In one embodiment, the nucleic acid is a cDNA encoding a peptide having an activity of caltrin. Preferably, the nucleic acid is a cDNA molecule comprising at least a portion of the nucleotide sequence encoding caltrin, shown in FIG. 1 (SEQ ID NO: 1) or FIG. 2 (SEQ ID NO: 2). A preferred portion of the cDNA molecules of FIG. 1 of FIG. 2 includes the coding region of the molecule.

In another embodiment, the nucleic acid of the invention encodes a peptide having an activity of caltrin and comprising an amino acid sequence shown in FIG. 3 (SEQ ID NO: 3) or FIG. 4 (SEQ ID NO: 4). Preferred nucleic acids encode a peptide having a caltrin activity and having at least about 50% identity, more preferably at least about 60% identity and most preferably at least about 70% identity with the sequence shown in FIG. 1 (SEQ ID NO: 1) or FIG. 2 (SEQ ID NO: 2). Nucleic acids which encode peptides having a caltrin activity and having at least about 90%, more preferably at least about 95%, and most preferably at least about 98-99% identity with a sequence set forth in FIG. 3 (SEQ ID NO: 3) or FIG. 4 (SEQ ID NO: 4) are also within the scope of the invention. Sequence identity refers to sequence similarity between two peptides having an activity of caltrin or between two nucleic acid molecules.

Isolated nucleic acids encoding peptides having an activity of caltrin, as described herein, and having a sequence which differs from the nucleotide sequences shown in FIG. 1 (SEQ ID NO: 1) or FIG. 2 (SEQ ID NO: 2) due to degeneracy in the genetic code are also within the scope of the invention. Such nucleic acids encode functionally equivalent peptides, but differ in sequence from the sequence of FIG. 1 or FIG. 2 due to degeneracy in the genetic code.

As discussed above, functionally active fragments of the nucleic acid encoding caltrin are also within the scope of the invention. As used herein, “a fragment” of the nucleic acid encoding caltrin refers to a nucleotide sequence having fewer nucleotides than the nucleotide sequence encoding the entire amino acid sequence of caltrin protein and which encodes a peptide having an activity of caltrin (ie., a peptide having at least one biological activity of caltrin) as defined herein.

Preferred nucleic acid fragments encode peptides of at least about 10 amino acid residues in length, preferably about 10-20 amino acid residues in length, and more preferably about 12-16 amino acid residues in length. Nucleic acid fragments which encode peptides having a caltrin activity of at least about 30 amino acid residues in length, at least about 40 amino acid residues in length, at least about 60 amino acid residues in length, at least about 80 amino acid residues in length, at least about 90 amino acid residues in length, and at least about 99 residues in length are also within the scope of this invention.

Nucleic acid fragments within the scope of the invention include those capable of hybridising under high or low stringency conditions as defined above with nucleic acids from other animal species for use in screening protocols to detect caltrin or other ligands that are cross-reactive with caltrin. Generally, the nucleic acid encoding a peptide having an activity of caltrin will be selected from the bases encoding the mature protein; however, in some instances it may be desirable to select all or part of a peptide from the leader sequence portion of the nucleic acids of the invention. Nucleic acids within the scope of the invention may also contain linker sequences, modified restriction endonuclease sites and other sequences useful for molecular cloning, expression or purification of recombinant peptides having an activity of caltrin.

A nucleic acid encoding a peptide having an activity of caltrin may be obtained from mRNA present in osteoblasts or osteoblast-like cells found in mammals. Nucleic acids encoding caltrin can also be isolated from mammalian genomic DNA. For example, the gene encoding caltrin can be cloned from either a cDNA or a genomic library in accordance with protocols herein described. A cDNA encoding caltrin can be obtained by isolating total mRNA from a mammal. Double stranded cDNAs can then be prepared from the total mRNA. Subsequently, the cDNAs can be inserted into a suitable plasmid or bacteriophage vector using any one of a number of known techniques. Genes encoding caltrin can also be cloned using established polymerase chain reaction (PCR) techniques in accordance with the nucleotide sequence information provided by the invention. The nucleic acids of the invention can be DNA or RNA. A preferred nucleic acid is a cDNA encoding caltrin having the sequence depicted in FIG. 1 (SEQ ID NO: 1) or FIG. 2 (SEQ ID NO: 2).

In another embodiment of the invention, nucleic acid sequences or functionally active fragments thereof which encode caltrin may be cloned in recombinant DNA molecules that direct expression of caltrin, or functionally active fragments thereof, in appropriate host cells. The host cell may be any prokaryotic or eukaryotic cell.

The nucleotide sequences of the present invention can be engineered using methods generally known in the art in order to alter caltrin-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

In another embodiment, sequences encoding caltrin may be synthesised, in whole or in part, using chemical methods well known in the art. (See, eg., Caruthers et al., 1980, Nucl. Acids Res. Symp. Ser. 215-223, and Horn et al., 1980, Nucl. Acids Res. Symp. Ser. 225-232). Alternatively, caltrin itself or functionally active fragments thereof may be synthesised using chemical methods. For example, peptide synthesis can be performed using various solid-phase techniques. (See, eg., Roberge et al., 1995, Science 269:202-204). Automated synthesis may be achieved using the ABI 431A Peptide Synthesiser (Perkin-Elmer). Additionally, the amino acid sequence of caltrin, or any part thereof, may be altered during direct synthesis and/or combined with sequences from other proteins, or any part thereof to produce a variant peptide. The peptide may be substantially purified by preparative high performance liquid chromatography. (See, eg, Chiez and Regnier, 1990, Methods Enzymol. 182:392-421). The composition of the synthetic peptides may be confirmed by amino acid analysis or by sequencing. (See. eg., Creighton (1984) Proteins. Structures and Molecular Properties. WH Freeman, New York N.Y.).

In order to express a biologically active caltrin, the nucleotide sequences encoding caltrin or functionally active variants or fragments thereof may be inserted into an appropriate expression vector, ie., a vector which contains the necessary elements for transcriptional and translational control of the inserted coding sequence in a suitable host. These elements include regulatory sequences, such as enhancers, constitutive and inducible promoters, and 5′ and 3′untranslated regions in the vector and in polynucleotide sequences encoding caltrin. Such elements may vary in their strength and specificity. Specific initiation signals may also be used to achieve more efficient translation of sequences encoding caltrin. Such signals include the ATG initiation codon and adjacent sequences, eg. the Kozak sequence. In cases where sequences encoding caltrin and its initiation codon and upstream regulatory sequences are inserted into the appropriate expression vector, no additional transcriptional or translational control signals may be needed. However, in cases where only coding sequence, or a functionally active fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used. (See, eg., Scharf et al., 1994, Results Probl. Cell Differ. 20: 125-162). Methods which are well known to those skilled in the art may be used to construct expression vectors containing sequences encoding caltrin and appropriate transcriptional and translational control elements. These methods include in vitro recombinant DNA techniques, synthetic techniques, and in vivo genetic recombination. (See, eg., Sambrook et al., supra; Ausubel et al., 1995, Current Protocols in Molecular Biology, John Wiley & Sons, New York N.Y., ch. 9, 13 and 16).

A variety of expression vector/host systems may be utilised to contain and express sequences encoding caltrin. These include, but are not limited to, microorganisms such as:
1). Bacteria transformed with recombinant bacteriophage, plasmid, or cosmid DNA expression vectors.
2). Yeast transformed with yeast expression vectors, for example, pYepSec1 (Baldari. et al., 1987, Embo J., 6:229-234), pMFa (Kurjan and Herskowitz, 1982, Cell, 30:933-943), pJRY88 (Schultz et al., 1987, Gene, 54:113-123), and pYES2 (Invitrogen Corporation, San Diego, Calif.).
3). Insect cell systems infected with viral expression vectors, for example, pAc series (Smith et al., 1983, Mol. Cell. Biol., 3:2156-2165) and the pVL series (Lucklow and Summers, 1989, Virology, 170:31-39).
3). Plant cell systems transformed with viral expression vectors, for example, cauliflower mosaic virus, CaMV, or tobacco mosaic virus, TMV or with bacterial expression vectors eg., Ti plasmids).
4). Animal cell systems such as COS cells (Gluzman, 1981, Cell, 23:175-182) used in conjunction with such vectors as pCDM 8 (Aruffo and Seed, 1987, Proc. Natl. Acad. Sci. USA, 84:8573-8577) for transient amplification/expression in mammalian cells, and CHO (dhfr− Chinese Hamster Ovary) cells used with vectors such as pMT2PC (Kaufman et al., 1987, EMBO J., 6:187-195) for stable amplification/expression in mammalian cells.

Expression in bacteria is most often carried out in E. coli and a number of cloning and expression vectors may be selected depending upon the use intended for nucleic acid sequence encoding caltrin. For example, routine cloning, subcloning, and propagation of nucleic acid sequences encoding caltrin can be achieved using a multifunctional E. coli vector such as pBLUESCRIPT™ (Stratagene, La Jolla Calif.) or pSPORTI™ plasmid (Life Technologies). Ligation of sequences encoding caltrin into the vector's multiple cloning site disrupts the lacZ gene, allowing a calorimetric screening procedure for identification of transformed bacteria containing recombinant molecules. In addition, these vectors may be useful for in vitro transcription, dideoxy sequencing, single strand rescue. In addition, such vectors direct either the secretion or intracellular retention of expressed proteins and enable integration of foreign sequences into the host genome for stable propagation. (See, eg., Ausubel, 1995, supra; Grant et al., 1987, Methods Enzymol. 153: 516-54; and Scorer et al., 1994, Bio/Technology 12: 181-184).

Either fusion or non-fusion inducible expression vectors may be used for the bacterial expression of caltrin. Typical fusion expression vectors include pGEX (AMRAD, Melbourne, Australia), PMAL (New England Biolabs, Beverly, Mass.) and pRIT5 (Pharmacia, Piscataway, N.J.) which fuse glutathione S-transferase, maltose E binding protein, or protein A, respectively, to the target recombinant protein.

Inducible non-fusion expression vectors include pTrc (Amann et al., 1988, Gene, 69:301-315) and pET 11d (Studier et al., 1990, Gene Expression Technology: Methods in Enzymology, 185, Academic Press, San Diego, Calif. 60-89). While target gene expression relies on host RNA polymerase transcription from the hybrid trp-lac fusion promoter in pTrc, expression of target genes inserted into pET 11d relies on transcription from the T7 gn10-lac 0 fusion promoter mediated by co-expressed viral RNA polymerase (T7 gn1). This viral polymerase is supplied by host strains BL21(DE3) or HMS174(DE3) from a resident λ prophage harbouring a T7 gn1 under the transcriptional control of the lacUV 5 promoter.

One strategy to maximise recombinant caltrin expression in E. coli is to express the protein in a host bacteria with an impaired capacity to proteolytically cleave the recombinant protein (Gottesman, 1990, Methods in Enzymology, 185, Academic Press, San Diego, Calif. 119-128). Another strategy would be to alter the nucleic acid encoding the caltrin protein to be inserted into an expression vector so that the individual codons for each amino acid would be those preferentially utilised in highly expressed E. coli proteins (Wada et al., 1992, Nuc. Acids Res., 20:2111-2118). Such alteration of nucleic acids of the invention can be carried out by standard DNA synthesis techniques.

Plant systems may also be used for expression of caltrin. Transcription of sequences encoding caltrin may be driven viral promoters, eg., the 35S and 19S promoters of CaMV used alone or in combination with the omega leader sequence from TMV (Takamatsu, 1987, EMBO J. 6: 307-311). Alternatively, plant promoters such as the small subunit of RUBISCO or heat shock promoters may be used (see, eg., Coruzzi et al., 1984, EMBO J. 3: 1671-1680; Broglie et al., 1984, Science 224: 838-843; and Winter et al., 1991, Results Probl. Cell Differ. 17:85-105). These constructs can be introduced into plant cells by direct DNA transformation or pathogen-mediated transfection (see, eg., The McGraw Hill Yearbook of Science and Technology (1992) McGraw Hill, New York N.Y., pp. 191-19). In mammalian cells, a number of viral-based expression systems may be utilised. In cases where an adenovirus is used as an expression vector, sequences encoding caltrin may be ligated into an adenovirus transcription/translation complex consisting of the late promoter and tripartite leader sequence. Insertion in a non-essential E1 or E3 region of the viral genome may be used to obtain infective virus which expresses caltrin in host cells (see, eg., Logan and Shenk, 1984, Proc. Natl. Acad. Sci. 81: 3655-3659). In addition, transcription enhancers, such as the Rous sarcoma virus (RSV) enhancer, may be used to increase expression in mammalian host cells. SV40 or EBV-based vectors may also be used for high-level protein expression. Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes (see, eg., Harrington et al., 1997, Nat. Genet. 15: 345-355).

For long term production of recombinant proteins in mammalian systems, stable expression of caltrin in cell lines is preferred. For example, sequences encoding caltrin can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells, which successfully express the introduced sequences. Resistant clones of stably transformed cells may be propagated using tissue culture techniques appropriate to the cell type. Any number of selection systems may be used to recover transformed cell lines. These include, but are not limited to, the herpes simplex virus thymidine kinase and adenine phosphoribosyltransferase genes, for use in tk− or apr cells, respectively (see, eg., Wigler et al., 1977, Cell 11: 223-232; Lowy et al., 1980 Cell 22: 817-823).

Also anti-metabolite, antibiotic or herbicide resistance can be used as the basis for selection. For example, dhfr confers resistance to methotrexate; neo confers resistance to the aminoglycosides, neomycin and G-418; and als or pat confer resistance to chlorsulfuron and phosphinotricin acetyltransferase, respectively (see, eg., Wigler et al., 1980, Proc. Natl. Acad. Sci. 77: 3567-3570; Colbere-Garapin et al., 1981, J. Mol. Biol. 150:1-14). Additional selectable genes have been described, eg., trpB and hisD, which alter cellular requirements for metabolites (see, eg., Hartman and Mulligan, 1988, Proc. Natl. Acad. Sci. 85: 8047-8051).

Visible markers, eg., anthocyanins, green fluorescent proteins (GFP; Clontech), β-glucuronidase and its substrate β-glucuronide, or luciferase and its substrate luciferin may be used. These markers can be used not only to identify transformants, but also to quantify the amount of transient or stable protein expression attributable to a specific vector system (see, eg., Rhodes, 1995, Methods Mol. Biol. 55: 121-131). Although the presence/absence of marker gene expression suggests that the gene of interest is also present, the presence and expression of the gene may need to be confirmed. For example, if the sequence encoding caltrin is inserted within a marker gene sequence, transformed cells containing sequences encoding caltrin can be identified by the absence of marker gene function. Alternatively, a marker gene can be placed in tandem with a sequence encoding caltrin under the control of a single promoter. Expression of the marker gene in response to induction or selection usually indicates expression of the tandem gene as well.

In general, host cells that contain the nucleic acid sequence encoding caltrin and that express caltrin may be identified by a variety of procedures known to those of skill in the art. These procedures include, but are not limited to, DNA-DNA or DNA-RNA hybridisations, PCR amplification, and protein bioassay or immunoassay techniques which include membrane, solution, or chip based technologies for the detection and/or quantification of nucleic acid or protein sequences. Immunological methods for detecting and measuring the expression of caltrin using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilising monoclonal antibodies reactive to two non-interfering epitopes on caltrin is preferred, but a competitive binding assay may be employed. These and other assays are well known in the art (see, eg., Hampton et al., 1990, Serological Methods. A Laboratory Manual. APS Press, St Paul Minn., Sect. IV; Coligan, supra). A wide variety of labels and conjugation techniques are known by those skilled in the art and may be used in various nucleic acid and amino acid assays. Means for producing labelled hybridisation or PCR probes for detecting sequences related to nucleic acid encoding caltrin include oligolabelling, nick translation, end-labelling, or PCR amplification using a labelled nucleotide.

Alternatively, the sequences encoding caltrin, or any functionally active fragments thereof, may be cloned into a vector for the production of a mRNA probe. Such vectors are known in the art, are commercially available, and may be used to synthesise RNA probes in vitro by addition of an appropriate RNA polymerase such as T7, T3, or SP6 and labelled nucleotides. These procedures may be conducted using a variety of commercially available kits, such as those provided by Amersham, Pharmacia Biotech, Promega (Madison Wis.), and US Biochemical. Suitable reporter molecules or labels, which may be used for ease of detection, include radionuclides, enzymes, fluorescent, chemiluminescent or chromogenic agents, as well as substrates, cofactors, inhibitors, magnetic particles and the like. Host cells transformed with nucleotide sequences encoding caltrin may be cultured under conditions suitable for the expression and recovery of the protein from cell culture. The protein produced by a transformed cell may be secreted or retained intracellularly depending on the sequence and/or the vector used.

As will be understood by those of skill in the art, expression vectors containing nucleic acids which encode caltrin may be designed to contain signal sequences which direct secretion of caltrin through a prokaryotic or eukaryotic cell membrane. In addition, a host cell strain may be chosen for its ability to modulate expression of the inserted sequences or to process the expressed protein in the desired fashion. Such modifications of the peptide include, but are not limited to acetylation, carboxylation, glycosylation, phosphorylation, lipidation, and acylation. Post-translational processing which cleaves a “prepro” form of the protein may also be used to specify protein targeting, folding, and/or activity. Different host cells which have specific cellular machinery and characteristic mechanisms for post-translational activities (eg., CHO, HeLa, MDCK, HEK293, and WI38), are available from the American Type Culture Collection (ATCC, Bethesda Md.) and may be chosen to ensure the correct modification and processing of the caltrin protein.

In another embodiment of the invention, natural, modified, or recombinant nucleic acid sequences encoding caltrin may be ligated to a heterologous sequence resulting in translation of a fusion protein in any of the aforementioned host systems. For example, a chimeric caltrin protein containing a heterologous moiety that can be recognised by a commercially available antibody may facilitate the screening of peptide libraries for inhibitors of caltrin activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilised glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognise these epitope tags.

A fusion protein may also be engineered to contain a proteolytic cleavage site located between the caltrin encoding sequence and the heterologous protein sequence, so that caltrin may be cleaved away from the heterologous moiety following purification. Methods for fusion protein expression and purification are discussed in Ausubel (1995, supra, ch 10). A variety of commercially available kits may also be used to facilitate expression and purification of fusion proteins. In a further embodiment of the invention, synthesis of radiolabelled caltrin may be achieved in vitro using the TNT rabbit reticulocyte lysate or wheat germ extract systems (Promega). These systems couple transcription and translation of protein-coding sequences operably associated with the T7, T3, or SP6 promoters. Translation takes place in the presence of a radiolabelled amino acid precursor, preferably ³⁵S-methionine. Fragments of caltrin may be produced not only by recombinant production, but also by direct peptide synthesis using solid-phase techniques (see, eg., Creighton, supra, pp. 55-60). Protein synthesis may be performed by manual techniques or by automation. Automated synthesis may be achieved, for example, using the ABI 431 A Peptide Synthesiser (Perkin-Elmer). Various fragments of caltrin may be synthesised separately and then combined to produce the full-length molecule.

As described briefly above, the present invention also provides expression vectors containing a nucleic acid encoding a peptide having an activity of caltrin, operably linked to at least one regulatory sequence. The term “operably linked” as used herein is intended to mean that the nucleotide sequence is linked to a regulatory sequence in a manner, which allows expression of the nucleotide sequence. Regulatory sequences are art-recognised and are selected to direct expression of the peptide having an activity of caltrin. Accordingly, the term regulatory sequence includes promoters, enhancers and other expression control elements. Such regulatory sequences are described in Goeddel; Gene Expression Technology: Methods in Enzymology 185, Academic Press, San Diego, Calif. (1990). It should be understood that the design of the expression vector might depend on such factors as the choice of the host cell to be transformed and/or the type of protein desired to be expressed. In one embodiment, the expression vector includes a DNA encoding a peptide having an activity of caltrin. Such expression vectors can be used to transfect cells to thereby produce proteins or peptides, including fusion proteins or peptides encoded by nucleic acids as described herein.

In one embodiment, the invention relates to isolated peptides, which have at least one biological activity of caltrin. For example, a peptide having an activity of caltrin may have the ability to elevate cytosolic calcium levels in osteoblasts and osteoblast-like cells. In another embodiment, peptides having a caltrin activity induce bone formation, which may include stimulation of osteoblasts or proliferation of osteoblasts. In yet another embodiment, a peptide having a caltrin activity has the ability to bind acrogranin (an acidic cysteine-rich glycoprotein of 67 kDa) and/or EMILIN.

A peptide having an activity of caltrin may differ in amino acid sequence from the caltrin sequence depicted in FIG. 3 (SEQ ID NO: 3) or FIG. 4 (SEQ ID NO: 4), but such differences result in a modified protein which functions in the same or similar manner as a native caltrin protein or which has the same or similar characteristics of a native caltrin protein. Various modifications of the caltrin protein to produce these and other functionally equivalent peptides are described in detail herein. The term “peptide,” as used herein, refers to peptides, proteins, and polypeptides.

A peptide can be produced by modification of the amino acid sequence of the caltrin protein shown in FIG. 3 (SEQ ID NO: 3) or FIG. 4 (SEQ ID NO: 4), such as a substitution, addition, or deletion of an amino acid residue which is not directly involved in the function of the protein. Peptides of the invention can be at least about 10 amino acid residues in length, preferably about 10-20 amino acid residues in length, and more preferably about 10-16 amino acid residues in length. Peptides having an activity of caltrin and which are at least about 30 amino acid residues in length, at least about 40 amino acid residues in length, at least about 60 amino acid residues in length, at least about 80 amino acid residues in length, and at least about 99 amino acid residues in length are also included within the scope of this invention.

Another embodiment of the invention provides a substantially pure preparation of a peptide having an activity of caltrin. Such a preparation is substantially free of proteins and peptides with which the peptide naturally occurs (ie., other mammalian peptides), either in a cell or when secreted by a cell.

The term “isolated” as used herein refers to a nucleic acid or peptide that is substantially free of cellular material or culture medium when produced by recombinant DNA techniques, or chemical precursors or other chemicals when chemically synthesised. Such proteins or peptides are also characterised as being free of all other mammalian proteins. Accordingly, an isolated peptide having an activity of caltrin is produced recombinantly or synthetically and is substantially free of cellular material and culture medium or substantially free of chemical precursors or other chemicals and is substantially free of all other mammalian proteins. An isolated nucleic acid is also free of sequences which naturally flank the nucleic acid (ie., sequences located at the 5′ and 3′ ends of the nucleic acid) in the organism from which the nucleic acid is derived.

Peptides having an activity of caltrin can be obtained, for example, by screening peptides recombinantly produced from the corresponding fragment of the nucleic acid of caltrin encoding such peptides. In addition, fragments can be chemically synthesised using techniques known in the art such as conventional Merrifield solid phase f-Moc or t-Boc chemistry. For example, the caltrin protein may be arbitrarily divided into fragments of desired length with no overlap of the fragments, or preferably divided into overlapping fragments of a desired length. The fragments can be produced (recombinantly or by chemical synthesis) and tested to identify those peptides having a caltrin activity (ie., the ability to elevate cytosolic calcium levels in osteoblasts and osteoblast-like cells or induce bone formation).

In one embodiment, peptides having an activity of caltrin can be identified by the ability of the peptide to bind acrogranin (an acidic cysteine-rich glycoprotein of 67 kDa) and/or EMILIN. These peptides are defined herein as comprising at least one acrogranin or EMILIN binding site.

Screening peptides for those which retain a caltrin activity as described herein can be accomplished using one or more of several different assays. For example, in vitro, caltrin peptides can be assayed for their ability to elevate cytosolic calcium levels in osteoblasts and osteoblast-like cells. For example, the FluoStar Optima fluorescence plate reader (BMG Lab Technologies, Offenburg, Germany) can be used to measure elevations in intracellular calcium. Briefly, COS-7, ST2 and primary calvarial osteoblasts can be seeded onto special black glass 96 well plates and grown overnight in a humidified incubator at 37° C. and 5% CO₂. Cells are then loaded with Fluo-3AM (10 μM) for 40 minutes prior to addition of caltrin. Fluo-3 is measured using an excitation filter of 485 nm and emission filter of 520 nm. Emission is measured from the bottom of the wells, while fluorescence is excited from the top.

Firstly, the internal calcium store is depleted by addition of thapsigargin. Calvarial osteoblasts are loaded with Fluo-3AM and then treated with 2 μM of thapsigargin. Once calcium is depleted, caltrin is subsequently added. Secondly, external calcium is removed by addition of EGTA. Calvarial osteoblastic cells are treated with 3 mM of EGTA to chelate calcium in the culture media. Fluo-3AM is subsequently loaded and cells are treated with caltrin. Thirdly, the Phospholipase C (PLC) pathway is examined. Calvarial osteoblasts are pre-treated with a PLC-inhibitor, U73122, for 10 minutes. Fluo-3AM is then loaded onto the cells and caltrin is added as per usual.

Alternatively, a common assay for demonstrating an ability to induce bone formation can be employed. For example, primary calvarial osteoblastic cells can be seeded onto 6 well plates and grown until confluent. Cell medium can then be exchanged with differentiation media (100 μg/mL Ascorbic Acid and 10 mM β-glycerophosphate) and the appropriate concentration of caltrin or dexamethasone added to each well. A single well can be left with normal media and untreated with caltrin (negative control). Fresh differentiation medium, caltrin protein and dexamethasone is replaced every second day for 21 days. To visualise mineralisation and calcium nodule formation, the cells are stained with Von Kossa (Schenk et al., 1984, In: Preparation of Calcified Tissues for Light Microscopy, G. R. Dickson, ed. Amsterdam, Elsevier). Briefly, cells are fixed with 4% paraformaldehyde (made in TBS). Cell medium is removed and washed three times with ddH₂0. Approximately, 500 μL of silver nitrate is added to each well in the dark for 15 minutes and subsequently exposed to UV light for 5 minutes. Cells are then treated with 5% sodium thiosulfate or Farmers Reducer for 5 minutes. Each well is then washed with ddH₂0 and air dried. Quantification can be done by scanning densitometry using the program ImageJ (http://rsb.info.nih.gov/ij/).

In another embodiment, a peptide having a caltrin activity is screened for the ability to bind to osteoblasts. For example, caltrin protein can be labelled using the lactoperoxidase method as described previously (Thorell & Johansson, 1971, Biochim Biophys Acta 251, 363-369). Briefly, 5 μg of recombinant caltrin protein can be added to lactoperoxidase and incubated for 30 minutes. After initiation of the labelling reaction by the addition of 1 mCi of Na¹²⁵I and hydrogen peroxide, the reaction can be halted using a stop reaction consisting of KI, albumin and sodium azide. The labelled protein sample can then be separated on a Sephadex G-50 column, eluted and the activity measured in an autogamma counter. Once labelled a qualitative binding analysis can be performed as follows:

Calvarial osteoblasts, ST2, COS-7, chondrocytes, RAW_{246.7} cells and RAW_{246.7} cell-derived osteoclasts can be seeded onto 12 well plates at a cell density of 2×10³ in 0.5 mL of culture media. When cells reach confluency, they can be washed and incubated with Blocking Buffer (serum-free growth media, 1% Fetal Bovine Serum and 50 mM HEPES), on ice for 10 minutes. Approximately long of labelled ¹²⁵I-caltrin, with or without a 75 fold excess of unlabelled caltrin, can be added to the appropriate wells. Cells are then incubated with caltrin for 2 hours at 4° C. Cells are then washed with cold PBS 5 times and solubilised with 0.2M NaOH and 1% SDS. The activity of each sample can then be measured using an autogamma counter.

Qualitative analysis of caltrin binding can also be undertaken using a saturation-binding curve and Scatchard analysis. To produce the saturation-binding curve, increasing concentrations of ¹²⁵I-caltrin, 5, 10, 20, 40, 80 and 160 ng can be added to 2×10⁵ primary mouse calvarial osteoblastic cells with or without excess unlabelled caltrin at 4° C. for 2 hours. The cells can be subsequently washed, solubilised and their activity measured. The specific binding is then calculated by subtracting the Non-Specific Binding (NSB) from the Total Binding counts. To produce a competition curve, calvarial osteoblastic cells can be incubated with increasing concentrations of unlabelled recombinant caltrin in the presence of long of ¹²⁵I-caltrin for 2 hours at 4° C. The competition curve, saturation binding curve, Scatchard plot and subsequent calculations of Kda and binding sites per cell can be produced using Graphpad Prism 4.0 software.

Peptides having an activity of caltrin which are to be used as therapeutic agents are preferably tested in mammalian models of bone disease, such as the lethal mouse model disclosed in Katagiri et al, 1998, Dev Genet. 22, 340-348, or in U.S. Pat. No. 6,660,468. Other animal models of bone diseases include the post menopause (eg. ovariectomised rat model) and the inflammation model (eg LPS-induced bone loss in mice).

It is possible to modify the structure of a peptide having an activity of caltrin for such purposes as increasing solubility, enhancing therapeutic or prophylactic efficacy, or stability (eg., shelf life ex vivo and resistance to proteolytic degradation in vivo). Such modified peptides are considered functional equivalents of peptides having an activity of caltrin as defined herein. A modified peptide can be produced in which the amino acid sequence has been altered, such as by amino acid substitution, deletion, or addition, to modify activity and/or reduce allergenicity, or to which a component has been added for the same purpose. Preferred amino acid substitutions for non-essential amino acids include, but are not limited to substitutions with alanine, glutamic acid, or a methyl amino acid.

Another example of modification of a peptide having an activity of caltrin is substitution of cysteine residues preferably with alanine, serine, threonine, leucine or glutamic acid residues to minimise dimerisation via disulfide linkages. In addition, amino acid side chains of fragments of the protein of the invention can be chemically modified. Another modification is cyclisation of the peptide.

In order to enhance stability and/or reactivity, a peptide having an activity of caltrin can be modified to incorporate one or more polymorphisms in the amino acid sequence of the caltrin protein resulting from any natural allelic variation. Additionally, D-amino acids, non-natural amino acids, or non-amino acid analogs can be substituted or added to produce a modified protein within the scope of this invention. Furthermore, a peptide having an activity of caltrin can be modified using polyethylene glycol (PEG) according to the method of Sehon and co-workers (Wie et al., supra) to produce a protein conjugated with PEG. In addition, PEG can be added during chemical synthesis of the protein. Other modifications of a peptide having an activity of caltrin include reduction/alkylation (Tarr, Methods of Protein Micro-characterisation, J. E. Silver ed., Humana Press, Clifton N.J. 155-194 (1986)); acylation (Tarr, supra); chemical coupling to an appropriate carrier (Mishell and Shiigi, eds, Selected Methods in Cellular Immunology, W H Freeman, San Francisco, Calif. (1980), U.S. Pat. No. 4,939,239; or mild formalin treatment (Marsh, 1971, Int. Arch. of Allergy and Appl. Immunol., 41:199-215).

Another aspect of the invention pertains to an antibody specifically reactive with a peptide having an activity of caltrin. The antibodies of this invention can be used to standardise caltrin extracts or to isolate the naturally-occurring or native form of caltrin. For example, by using peptides having an activity of caltrin based on the cDNA sequence of caltrin, anti-protein/anti-peptide antisera or monoclonal antibodies can be made using standard methods. A mammal such as a mouse, a hamster or rabbit can be immunised with an immunogenic form of the peptide (eg., caltrin protein or an antigenic fragment, which is capable of eliciting an antibody response). Techniques for conferring immunogenicity on a protein or peptide include conjugation to carriers or other techniques well known in the art. A peptide having an activity of caltrin can be administered in the presence of adjuvant. The progress of immunisation can be monitored by detection of antibody titres in plasma or serum. Standard ELISA or other immunoassay can be used with the immunogen as antigen to assess the levels of antibodies.

Following immunisation, anti-caltrin antisera can be obtained and, if desired, polyclonal anti-caltrin antibodies isolated from the serum. To produce monoclonal antibodies, antibody producing cells (lymphocytes) can be harvested from an immunised animal and fused by standard somatic cell fusion procedures with immortalising cells such as myeloma cells to yield hybridoma cells. Such techniques are well known in the art, for example the hybridoma technique originally developed by Kohler and Milstein, 1975, Nature, 256:495-497; as well as other techniques such as the human B cell hybridoma technique (Kozbar et al., 1983, Immunology Today, 4:72; and the EBV-hybridoma technique to produce human monoclonal antibodies (Cole et al., 1985, Monoclonal Antibodies and Cancer Therapy, Alan R. Liss, Inc. pp. 77-96). Hybridoma cells can be screened immunochemically for production of antibodies specifically reactive with a peptide having an activity of caltrin and the monoclonal antibodies isolated.

The term “antibody” as used herein is intended to include fragments thereof which are also specifically reactive with the peptide having an activity of caltrin. Antibodies can be fragmented using conventional techniques and the fragments screened for utility in the same manner as described above for whole antibodies. For example, F(ab′)₂ fragments can be generated by treating antibody with pepsin. The resulting F(ab′)₂ fragment can be treated to reduce disulfide bridges to produce Fab′ fragments. The antibody of the present invention is further intended to include bispecific and chimeric molecules having an anti-caltrin portion.

The caltrin peptides and fragments thereof of the present invention can also be used in specific assays related to the functional information provided in the following examples. They can be used to as reagents (including labelled reagents) in assays designed to quantitatively determine levels of the caltrin (or its binding partner or ligand) in biological fluids. Caltrin peptides and fragments can also be used as markers for tissues in which the corresponding protein is preferentially expressed (either constitutively or at a particular stage of tissue differentiation or development or in a disease state). Where the caltrin protein binds or potentially binds to another protein or ligand, the caltrin can then be used to identify the binding partner/ligand so as to develop a system to identify modulators of the binding interaction. Any or all of these uses are capable of being developed into reagent grade or kit format for commercialisation as commercial products.

Methods for performing the uses listed above are well known to those skilled in the art. References disclosing such methods include “Sambrook supra and “Methods in Enzymology: Guide to Molecular Cloning Techniques”, Academic Press, Berger, S. L. and A. R. Kimmel eds., 1987.

The terms “subject” or “individual” are used interchangeably herein to refer to any member of the subphylum cordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The terms do not denote a particular age. Thus, both adult and newborn individuals are intended to be covered. The methods described herein are intended for use in any of the above vertebrate species, since the immune systems of all of these vertebrates operate similarly.

Thus, provided is the treatment of mammals such as humans, as well as those mammals of economical importance and/or social importance to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, eg., poultry, such as turkeys, chickens, ducks, geese, guinea fowl, and the like, as they are also of economical importance to humans. Thus, provided is the treatment of livestock, including, but not limited to, domesticated swine (pigs and hogs), ruminants, horses, poultry, and the like.

A peptide having an activity of caltrin when administered to a subject requiring treatment for a bone disorder or disease is capable of modifying osteoblast function in the subject. As used herein, modification of the osteoblast function of a subject can be defined as an increase in cytosolic calcium levels or diminution in symptoms of the bone disorder or disease, as determined by standard clinical procedures. As referred to herein, a diminution in symptoms includes any reduction in the symptoms of bone disease in a subject following a treatment regimen with a peptide of the invention. This diminution in symptoms may be determined subjectively (eg., the patient feels more comfortable upon exposure to the caltrin), or clinically, such as with a standard bone densitometry test.

The present invention further provides methods of preventing and/or treating bone disease or disorders in a subject. Generally, the terms “treating”, “treatment” and the like are used herein to mean affecting an individual or subject, their tissue or cells to obtain a desired pharmacological and/or physiological effect. The effect may be prophylactic in terms of completely or partially preventing the bone disorder or disease or sign or symptom thereof, and/or may be therapeutic in terms of a partial or complete cure of the bone disorder or disease. “Treating” as used herein covers any treatment of, or prevention of bone disorder or disease in a vertebrate, a mammal, particularly a human, and includes: (a) preventing the bone disorder or disease from occurring in a subject that may be predisposed to the bone disorder or disease, but has not yet been diagnosed as having it; (b) inhibiting the bone disorder or disease, ie., arresting its development; or (c) relieving or ameliorating the symptoms of the bone disorder or disease, ie., cause regression of the symptoms of the disorder or disease.

The bone disorder is preferably characterised by excessive osteoclastic bone resorption and/or hypercalcemic serum effects. As such the term “bone disorder” as used herein means the need for bone repair or replacement. Conditions in which the need for bone repair or replacement may arise include: osteoporosis (including post menopausal osteoporosis, male and female senile osteoporosis and corticosteroid induced osteoporosis), osteoarthritis, Paget's disease, osteomalacia, multiple myeloma and other forms of cancer, prolonged bed rest, chronic disuse of a limb, anorexia, microgravity, exogenous and endogenous gonadal insufficiency, bone fracture, non-union, defect, prosthesis implantation and the like.

In order to bring about the treatment of the bone disorder or disease, an effective amount of caltrin peptide, or a peptide having an activity of caltrin is administered to a subject in need of therapy. The term “effective amount of caltrin peptide” as used herein means that the caltrin peptide is sufficient to produce an effect on the osteoblasts. For example, in one embodiment caltrin would be administered in an “effective amount” so that the cytosolic calcium levels in osteoblasts or osteoblast-like cells increased.

Pharmaceutical compositions comprising a caltrin according to embodiments of the present invention are also provided. The caltrin as described above may be formulated for administration in a pharmaceutical carrier in accordance with known techniques. See, eg., Remington, The Science And Practice of Pharmacy (9^{th} Ed. 1995). In the manufacture of a pharmaceutical composition according to embodiments of the present invention, the caltrin is typically admixed with, inter alia, a pharmaceutically acceptable carrier. The carrier must, of course, be acceptable in the sense of being compatible with any other ingredients in the pharmaceutical composition and should not be deleterious to the subject being treated. The carrier may be a solid or a liquid, or both, and is preferably formulated with the caltrin as a unit-dose formulation, for example, a tablet, which may contain from about 0.01 or 0.5% to about 95% or 99% by weight of the caltrin. The pharmaceutical compositions may be prepared by any of the well-known techniques of pharmacy including, but not limited to, admixing the components, optionally including one or more accessory ingredients.

The pharmaceutical compositions according to embodiments of the present invention include those suitable for oral, rectal, topical, inhalation (eg., via an aerosol) buccal (eg., sub-lingual), vaginal, parenteral (eg., subcutaneous, intramuscular, intradermal, intraarticular, intrapleural, intraperitoneal, intracerebral, intraarterial, or intravenous), topical (ie., both skin and mucosal surfaces, including airway surfaces) and transdermal administration, although the most suitable route in any given case will depend on the nature and severity of the condition being treated and on the nature of the particular caltrin or caltrin peptide which is being used.

Pharmaceutical compositions suitable for oral administration may be presented in discrete units, such as capsules, cachets, lozenges, or tablets, each containing a predetermined amount of the caltrin; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the caltrin and a suitable carrier (which may contain one or more accessory ingredients as noted above). In general, the pharmaceutical composition according to embodiments of the present invention are prepared by uniformly and intimately admixing the caltrin with a liquid or finely divided solid carrier, or both, and then, if necessary, shaping the resulting mixture. For example, a tablet may be prepared by compressing or moulding a powder or granules containing the mixture of caltrin and pharmaceutically acceptable carrier, optionally with one or more accessory ingredients. Compressed tablets may be prepared by compressing, in a suitable machine, the mixture in a free-flowing form, such as a powder or granules optionally mixed with a binder, lubricant, inert diluent, and/or surface active/dispersing agent(s). Moulded tablets may be made by moulding, in a suitable machine, the powdered compound moistened with an inert liquid binder.

Pharmaceutical compositions suitable for buccal (sub-lingual) administration include lozenges comprising the caltrin in a flavoured base, usually sucrose and acacia or tragacanth; and pastilles comprising the caltrin in an inert base such as gelatin and glycerin or sucrose and acacia.

Pharmaceutical compositions according to embodiments of the present invention suitable for parenteral administration comprise sterile aqueous and non-aqueous injection solutions of the caltrin, which preparations are preferably isotonic with the blood of the intended recipient. These preparations may contain anti-oxidants, buffers, bacteriostats and solutes which render the composition isotonic with the blood of the intended recipient. Aqueous and non-aqueous sterile suspensions may include suspending agents and thickening agents. The compositions may be presented in unit/dose or multi-dose containers, for example sealed ampoules and vials, and may be stored in a freeze-dried (lyophilised) condition requiring only the addition of the sterile liquid carrier, for example, saline or water-for-injection immediately prior to use. Extemporaneous injection solutions and suspensions may be prepared from sterile powders, granules and tablets of the kind previously described. For example, an injectable, stable, sterile composition comprising a caltrin in a unit dosage form in a sealed container may be provided. The caltrin is provided in the form of a lyophilisate, which is capable of being reconstituted with a suitable pharmaceutically acceptable carrier to form a liquid composition suitable for injection thereof into a subject. The unit dosage form typically comprises from about 10 mg to about 10 grams of the caltrin. When the caltrin is substantially water-insoluble, a sufficient amount of emulsifying agent, which is physiologically acceptable may be employed in sufficient quantity to emulsify the caltrin in an aqueous carrier. One such useful emulsifying agent is phosphatidyl choline.

Pharmaceutical compositions suitable for rectal administration are preferably presented as unit dose suppositories. These may be prepared by admixing the caltrin with one or more conventional solid carriers, for example, cocoa butter, and then shaping the resulting mixture.

Pharmaceutical compositions suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Pharmaceutical compositions suitable for transdermal administration may be presented as discrete patches adapted to remain in intimate contact with the epidermis of the recipient for a prolonged period of time. Compositions suitable for transdermal administration may also be delivered by iontophoresis (see, for example, Pharmaceutical Research 3 (6):318 (1986)) and typically take the form of an optionally buffered aqueous solution of the caltrin. Suitable formulations comprise citrate or bis/tris buffer (pH 6) or ethanol/water and contain from 0.1 to 0.2 M active ingredient.

Methods of treating a bone disorder in a subject in need of such treatment by administering an effective amount of such pharmaceutical compositions are also provided. The bone disorder is preferably characterised by excessive osteoclastic bone resorption and/or hypercalcemic serum effects. Bone disorders that may be treated and/or prevented by methods of the present invention include, but are not limited to, osteoporosis, Paget's disease, and hypercalcemia.

The effective amount of any mixture of caltrin, the use of which is in the scope of present invention, will vary somewhat from mixture to mixture, and patient to patient, and will depend upon factors such as the age and condition of the patient and the route of delivery. Such dosages can be determined in accordance with routine pharmacological procedures known to those skilled in the art. As a general proposition, a dosage from about 0.1 to about 50 mg/kg will have therapeutic efficacy, with all weights being calculated based upon the weight of the mixture of caltrin. Toxicity concerns at the higher level may restrict intravenous dosages to a lower level such as up to about 10 mg/kg, with all weights being calculated based upon the weight of the active base. A dosage from about 10 mg/kg to about 50 mg/kg may be employed for oral administration. Typically, a dosage from about 0.5 mg/kg to 5 mg/kg may be employed for intramuscular injection. The frequency of administration is usually one, two, or three times per day or as necessary to control the condition. Alternatively, the caltrin may be administered by continuous infusion. The duration of treatment depends on the type of bone disorder being treated and may be for as long as the life of the patient.

Throughout the specification, unless the context requires otherwise, the word “comprise” or variations such as “comprises” or “comprising”, will be understood to imply the inclusion of a stated integer or group of integers but not the exclusion of any other integer or group of integers.

The invention will now be further described by way of reference only to the following non-limiting examples. It should be understood, however, that the examples following are illustrative only, and should not be taken in any way as a restriction on the generality of the invention described above. In particular, while the invention is described in detail in relation to the use of a specific caltrin peptide, it will be clearly understood that the findings herein are not limited to this peptide.

### EXAMPLE 1

### Identification of a Novel Osteoclast-Derived Factor in Osteoclasts

A cDNA-subtracted library was constructed according to the procedures of the ClonTech PCR-Select Subtraction Kit (California, USA). Briefly, double-stranded cDNA was prepared from 2 μg of poly(A)⁺ RNA obtained from the haematopoietic macrophage cell line RAW_{246.7} (Xu et al., 2000, J. Cell Biochem. 88, 1256-1264) treated with (tester) and without (driver) 100 ng/mL of RANKL for 5 days. Tester and driver cDNAs were digested with RsaI to generate shorter, blunt-ended double stranded cDNA fragments for optimal subtraction. The tester, but not the driver cDNA, was then treated with adaptors. Both tester and driver were subject to hybridisation, and primary PCR was employed to amplify differentially expressed sequences. The subtraction was also performed with RANKL-treated RAW_{246.7} cells as the driver, and untreated RAW_{246.7} cells as the tester to produce a reverse-subtracted cDNA pool. The forward-subtracted PCR products were sub-cloned into a pCR2.1 T/A cloning vector (Invitrogen, Carlsbad, Calif.) to generate a subtracted cDNA library. Individual clones were isolated and screened using the PCR-select differential screening protocol (ClonTech). Briefly, cDNA clones isolated from a subtracted cDNA library were arrayed onto the Hybond-N+ membrane in duplicates (Amersham-Pharmacia BioTech, Sydney, Australia). Membranes were hybridised either with tester, driver, or subtracted cDNA probes that had been radiolabelled with ³²P-dCTP (Geneworks, Adelaide, Australia) using random priming duplicates (Amerhsam-Pharmacia Biotech, Sydney, Australia). Membranes were dried and exposed to autoradiographic film (Kodak). The cDNA sequence of differentially expressed clones was determined using BigDye termination Reaction Mix and an automated sequencer (Perkin-Elmer, Foster City, Calif.).

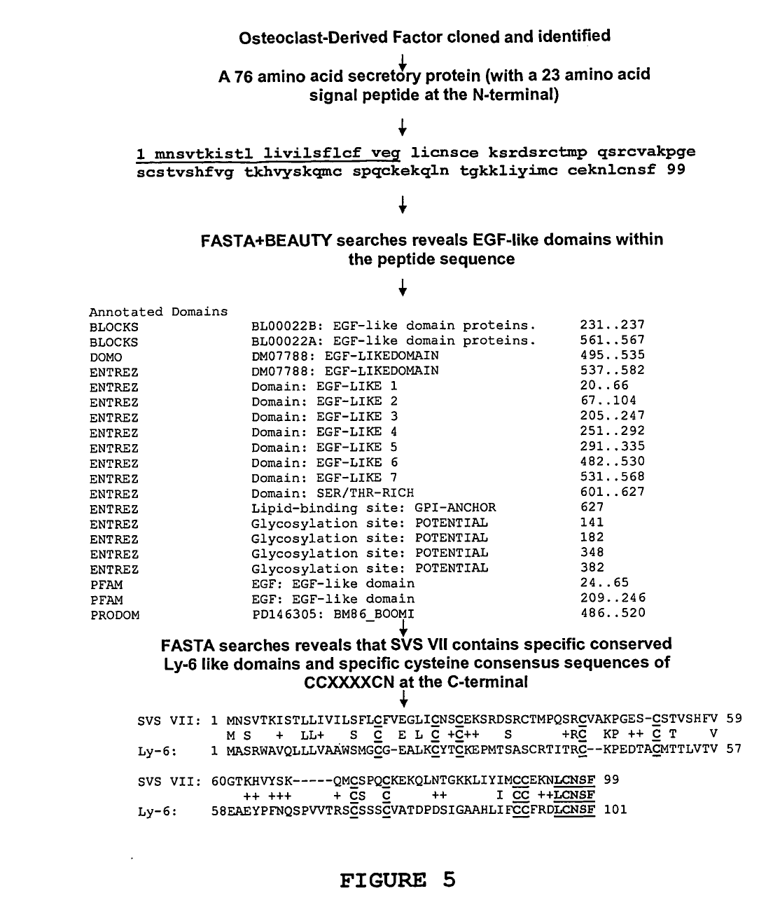

A number of cDNA clones were isolated and sequenced and searched against a database (BLAST—www.ncbi.nlm.gov/BLAST) of known genes. One cDNA clone encoding a small cysteine rich secretory protein called calcium transport inhibitor, caltrin, was identified. Further bioinformatic searches by FASTA and BEAUTY against several protein databases (FIG. 5) revealed the presence of epidermal growth factor (EGF)-like domains, suggesting that the promotion of cellular growth may be a potential function for caltrin. Additionally, this protein has a secretory signal and specific conserved cysteine residues that belong to the Lymphocyte-6 (Ly-6) antigen superfamily; a family of cytoplasmic and secreted proteins involved in growth, proliferation, differentiation and survival of leucocytes.

This protein was later renamed Osteoclast-Derived Osteoblastic Factor (ODOF).

### EXAMPLE 2

### Characterisation of the Expression Pattern of a Novel Caltrin

Once caltrin was identified in osteoclasts using subtractive hybridisation, the next step was to examine the level of caltrin mRNA in osteoclasts and other cells and tissues. To assess the level of caltrin mRNA expression during osteoclastogenesis, RAW_{246.7} cells were cultured in the presence or absence of soluble RANKL and subjected to semi-quantitative RT-PCR analysis.

Briefly, total RNA was isolated from cultured cells using RNAzol solution according to the manufacturer's instructions (Ambion Inc., Austin, Tex.). For RT-PCR, single stranded cDNA was prepared from 2 μg of total RNA using reverse transcriptase with an oligo-dT primer. Two microlitres of each cDNA was subjected to 30 cycles of PCR (94° C., 40 sec; 54° C., 40 sec; and 72° C., 40 sec) using mouse caltrin specific primers 5′ATGATTCAGTGACGAAAT3′; and 5′GAAGCTATTACACAAGTTTT3′. To examine whether caltrin was expressed in osteoblasts, total RNA was isolated from primary calvarial cells and ST2-osteoblast like cells and was subsequently subjected to semi-quantitative RT-PCR, as discussed above.

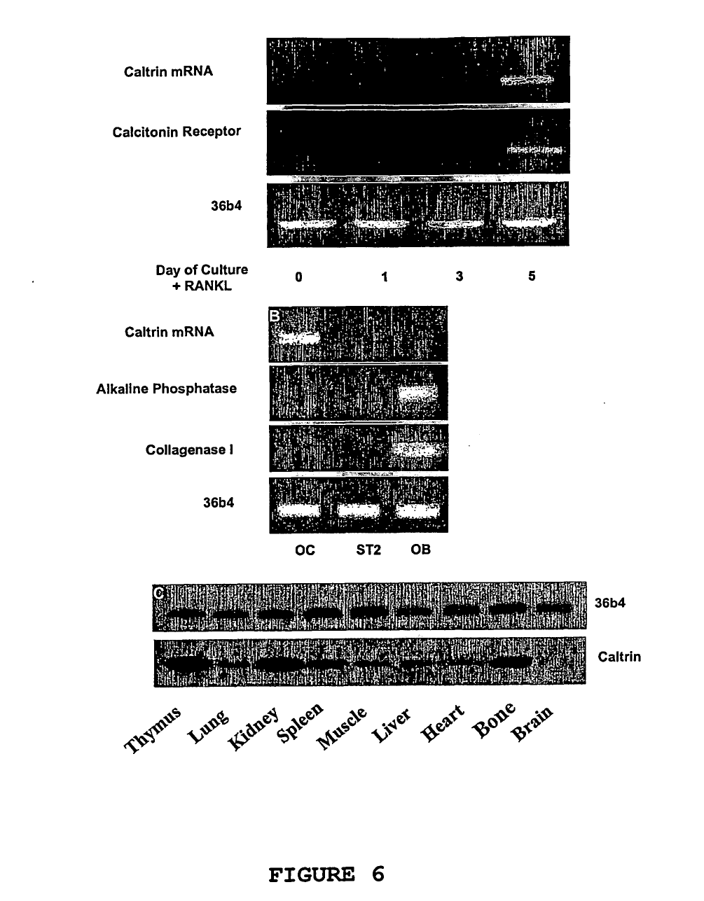

As shown in FIG. 6A, caltrin mRNA was upregulated during osteoclastogenesis, with specific high expression in day 5 of differentiation; the time-point when precursor cells differentiate into proper osteoclasts. Moreover, the presence of the calcitonin receptor on day 5 of differentiation confirms the formation of osteoclasts in culture. The expression of 36B4 demonstrates equal cDNA levels for each sample. These observations indicate that caltrin is differentially expressed in osteoclasts.

As can be observed in FIG. 6B, primary calvarial osteoblasts and ST2 cells do not express the caltrin gene. The expression of 36B4 was again used as an internal control. These observations indicate that osteoclastic cells, but not osteoblasts, express caltrin. To appraise the expression of caltrin mRNA in various snap-frozen mouse tissues, total RNA was isolated using RNAzol solution as described above and subjected to RT-PCR. FIG. 6C shows that caltrin expression was highest in bone, thymus and kidney, but weakly present in all tested mouse tissues. Again, the expression of 36B4 was also used as an internal control. It is noteworthy to mention that caltrin has only been characterised in spermatozoa and not in any other cell or tissue type.

### EXAMPLE 3

### Expression of Recombinant Caltrin Using the Baculovirus Expression Vector System (BEVS)

In order to characterise the function of caltrin in bone, the BEVS (Kitts, 1995, Methods Mol Biol 39, 129-142) was utilised to produce active recombinant his-tagged proteins that can be used in subsequent in vitro and in vivo experiments.

Briefly, total RNA was isolated from RAW_{264.7} cell-derived osteoclasts. For RT-PCR, single-stranded cDNA was prepared from 2 μg of total RNA using reverse transcriptase and an oligo-dT primer. Two μl of cDNA was subject to 30 cycles of PCR amplification (94° C., 40 sec; 54° C., 40 sec; and 72° C., 40 sec) using the caltrin specific primers 5′ATGATTCAGTGACGAAAT3′ (forward) and 5′GAAGCTATTACACAAGTTTT3′ (reverse). The amplified product was gel purified on a 1% TAE-agarose gel and extracted using the QIAEX II gel purification kit (Qiagen Inc, Valenica, Calif.). The PCR products were cloned into the TOPO TA Cloning site of pcDNA3.1/V5/His-TOPO (Invitrogen, Carlsbad, Calif.). The resulting plasmid, pcDNA3.1/V5/His-caltrin was confirmed by sequence analysis using the T7 primer, 5′TAATACGACTCACTATAGGG3′.

The pcDNA3.1/V5/His-caltrin vector was subsequently PCR-amplified with the T7 and BGH, 5′TAGAGGCACAGTCGAGG3′, primers. The PCR products were cloned into the pGEM-T-Easy T/A cloning vector. The resulting plasmid pGEM-T-caltrin was digested with SalI, blunt-ended with T4 DNA polymerase, and then with KpnI. The caltrin cDNA insert was ligated into the KpnI/StuI site of pBacPAK9 transfer vector (Clontech) to make pBacPAK9-caltrin. As before, the identity of the recombinant plasmid was confirmed by DNA sequencing.

Spodoptera frugiperda 9 (Sf-9) cells (Kitts supra) were co-transfected at 27° C. with 500 ng of the transfer vector pBacPak9-caltrin and 2 μg of linearised Bsu36 1 digested BackPak6 Viral DNA according to Clontech protocols. The caltrin gene was then propagated with the baculovirus, Autographa californica. The recombinant baculovirus was then isolated and amplified by using the BacPak BEVS Kit (Clontech). Optimal time for harvest was 72 hours post-infection and the Multiplicity of Infection (M.O.I.) was 5.

Sf-9 cells were cultured in serum free medium at 27° C. within a 1000 RPM shaking incubator according to the supplier's instructions (Gibco-BRL). The Coulter Counter Model ZM counting chamber and Trypan Blue stain were utilised to determine cell number and viability, respectively.

Purification of recombinant caltrin was either by Co²⁺-CmAsp resin or nickel (Ni²⁺)-nitrilotriacetic acid (NTA) resin. The Co²⁺-CmAsp resin involved a hybrid batch/gravity flow procedure (Clontech). In this technique pre-equilibration, binding and most of the washing was performed in a batch format, while additional washing and elution was done on a chromatography column utilising gravity flow. Binding, washing and elution conditions were optimised to allow the purification of caltrin at the highest yield and homogeneity. Briefly, the Co²⁺-CmAsp Resin was pre-equilibrated with 1× Extraction/Wash Buffer consisting of 50 mM NaH₂PO₄ and 300 mM NaCl at pH 7.0. After optimal infection the Sf-9 cells were removed and centrifuged at 6000 RPM at 4° C. in a Beckman JA14 rotor for 10 minutes. The supernatant was collected and incubated with pre-equilibrated Co²⁺ resin with gentle agitation at 4° C. for 2 hours. The resin was then centrifuged at 3000 RPM for 5 minutes and washed twice with 50 mL 1× Extraction/Wash Buffer containing 20 mM imidazole. The resin was then transferred to a Poly-Prep Biorad Chromatography column where the resin was washed with 10 mL of 1× Extraction/Wash Buffer containing 20 mM imidazole. Recombinant caltrin was eluted by the addition of 2 mL of 1× Elution Buffer consisting of 50 mM NaH₂PO₄, 300 mM NaCl and 150 mM imidazole. Aliquots of 500 μL were collected and judged homogenous by SDS-PAGE (Laemnli, 1970, Nature 227, 680-685).

Purified recombinant caltrin was dialysed overnight in phosphate-buffered saline (PBS) and concentrated by ultrafiltration at 4° C. with YM3 filter (Mᵣ cut-off at 3000; Millipore, USA). The concentration was calculated using the BCA Assay (Smith et al., 1985, Anal Biochem 150, 76-85) with BSA as a standard. 200 mL of caltrin-infected Sf-9 cell supernatant yielded 2 mg/mL of recombinant caltrin.

Affinity purification using the Ni²⁺-NTA Resin followed a similar procedure to the Co²⁺-CmAsp Resin. Binding, washing and elution conditions were optimised to allow the purification of caltrin at the highest yield and homogeneity. Briefly, the Ni²⁺-NTA resin was pre-equilibrated with 1× Binding Buffer consisting of 20 mM NaH₂PO₄, 500 mM NaCl, pH 7.4. The insect cells were centrifuged at 6000 RPM at 4° C. in a Beckman JA14 rotor for 10 minutes. The supernatant was incubated with pre-equilibrated Ni²⁺-NTA Resin for 2 hours with gentle agitation at 4° C. The resin was then centrifuged at 3000 RPM for 5 minutes and washed twice with 10 mL of 1× Wash Buffer consisting of 10 mM NaH₂PO₄, 500 mM NaCl, 10 mM imidazole, pH 6.0. The resin was then transferred to a Poly-Prep Biorad Chromatography column. Recombinant caltrin was eluted by an increasing imidazole concentration gradient of 200 mM, 400 mM, 600 mM, 800 μM and 1 M. The 1× Elution Buffer consisted of 20 mM NaH₂PO₄ and 500 mM NaCl with the appropriate imidazole concentration at pH 6.0. Homogeneity of the eluted protein was again tested by SDS-PAGE.

Recombinant caltrin protein was separated by SDS-PAGE and transferred to a nitrocellulose membrane by electroblotting as previously described (Sambrook et al., supra). Blots were incubated in 5% dry skim milk in tris buffered saline-Tween (TBS-Tween) for 1 hour and subsequently washed twice with TBS-Tween for 5 minutes. The blots were then probed with either anti-His (1:3000), anti-V5 (1:10000) or anti-caltrin antibody (1:5000) in TBS-Tween containing 1% skim milk. Blots were washed three times for 5 minutes with TBS-Tween, and incubated with either anti-Mouse or anti-Rabbit peroxidase conjugated IgG (Vector Laboratories, CA) at a final dilution of 1:5000 in TBS-Tween containing 1% skim milk. Finally, the blots were washed twice with TBS-Tween for five minutes and twice with TBS for five minutes. The antibody reactivity was detected by the Enhanced Chemiluminescence System (ECL) according to the manufacturer's instructions (Amersham).

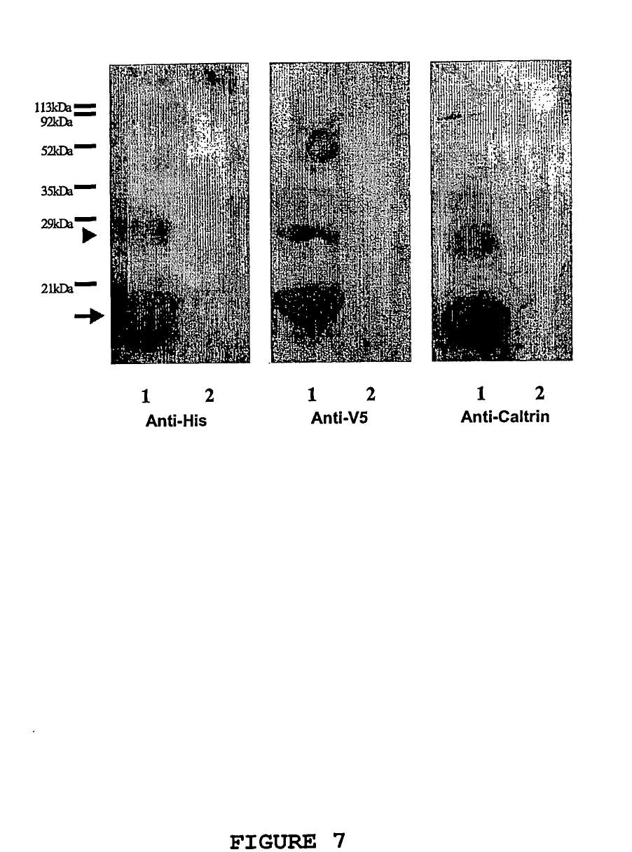

FIG. 7 shows a western blot analysis using an anti-His monoclonal antibody. It can be seen that a 14.5 kDa protein (arrow) corresponding to the size of recombinant caltrin was seen. The same results were demonstrated when employing the anti-V5 and anti-caltrin antibodies. The presence of a 14.5 kDa band in the insect cell supernatant suggests that caltrin is a secreted protein.

### EXAMPLE 4

### Qualitative Binding of Caltrin

The binding of the recombinant caltrin produced in Example 3 was initially examined using a fluorescent based binding technique, which has been used in various studies (Foley et al., 2003, Eur J Biochem 270, 3610-3618). In this methodology, a protein-antibody bridge is formed, similar to the approach used in a western blot analysis, but with the antibody conjugated to a specific fluorescent probe. Protein is added to the target cells, followed by a primary antibody, specific for the protein, and a secondary antibody, specific for the primary, attached to a fluorescent probe. The cell-to-protein suspension is then examined using Fluorescence Activated Cell Sorting (FACS) analysis.

The FACS-based binding approach was used to qualitatively evaluate the interaction of ST2-osteoblast like cells, COS-7 monkey kidney cells and RAW_{246.7} cells to caltrin recombinant protein. In addition, primary anti-caltrin and secondary anti-rabbit IgG antibody conjugated to Alexa 488 were also used in this assay.

Briefly, RAW_{246.7}, COS-7 and ST2-osteoblast like cells at a density of 1×10⁶ were cultured in eppendorf tubes and washed with PBS. Cells were resuspended in 20 μL of PBS and incubated at 4° C. for 1 hour after addition of 2 μg caltrin. After incubation, the cells were washed, resuspended in 20 μl of PBS and treated with anti-caltrin ( 1/100) antibody for 1 hour at 4° C. Cells were then washed three times with 500 μL of PBS and final resuspension was done in 20 μl of the same buffer. Anti-Rabbit-FITC, specific to the primary antibody, was added to each sample and incubated for 30 minutes at 4° C. Finally, the cells were washed and resuspended in 1 mL of PBS. Cell binding was subsequently analysed on a FACSVantage machine.

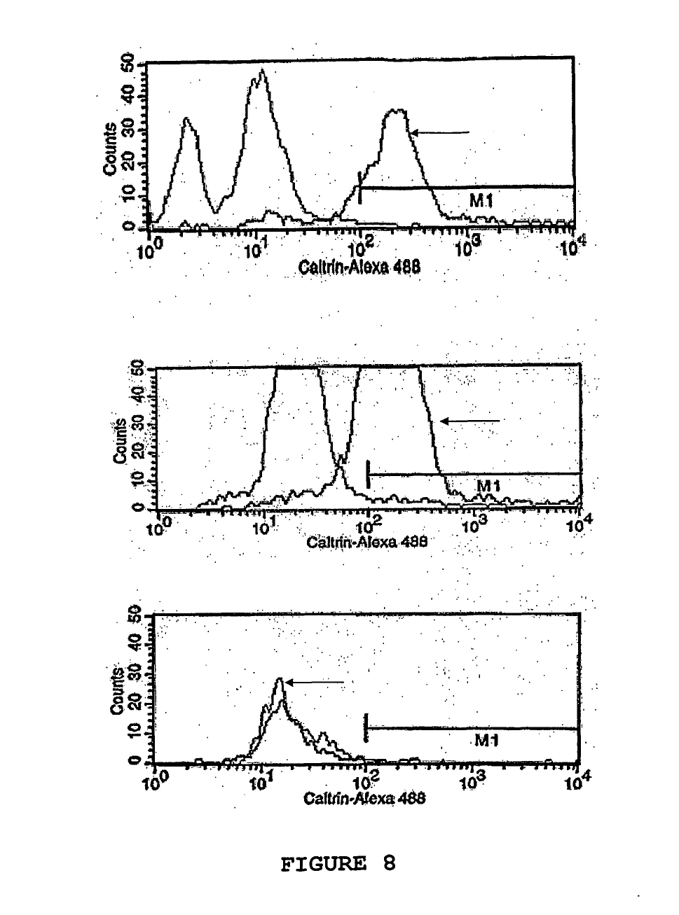

FIG. 8A shows the fluorescence curve for COS-7 cells in the presence and absence of recombinant caltrin. In the absence of recombinant protein (black lines), only the primary and secondary antibodies exist and a fluorescence curve can be observed corresponding to the non-specific binding of the antibodies to the cells. However, in the presence of caltrin protein (red lines/arrow) there is a significant shift (approximately 80%) in the mean fluorescence, indicating substantial binding to COS-7 cells.

For the binding between ST2 cells and caltrin, the FACS-based approach reveals a considerable shift (approximately 75%) in the mean fluorescence (FIG. 8B, red line/arrow) compared to the cells without recombinant protein (black line), indicating strong cell-to-protein interaction. Interestingly, when RAW_{246.7} cells were used, no significant shift in mean fluorescence was seen, indicating a lack of binding between this cell line and caltrin (FIG. 8C). Together, these observations indicate that caltrin binds to osteoblast-like cells, but not to osteoclastic pre-cursors. However, the FACS-based binding technique had two main disadvantages that made analysis difficult. The first was a high, non-specific fluorescence binding background, and the second was the requirement of a significantly high cell number (1×10⁶) to produce reliable results. The latter case meant that cells such as osteoclasts could not be used.

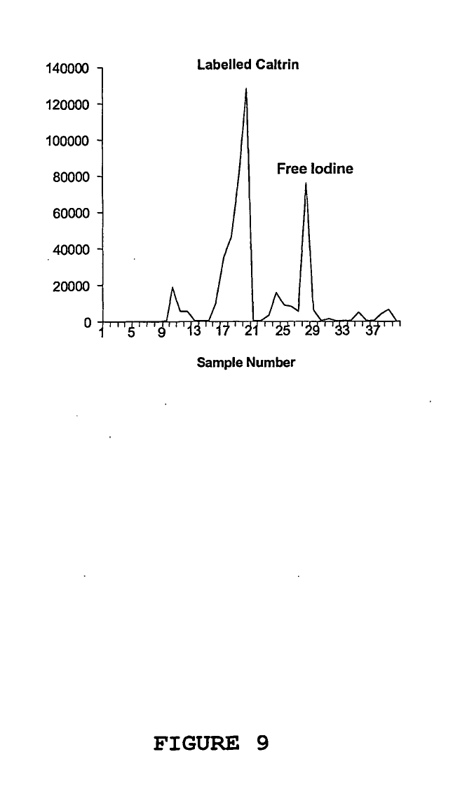

An iodine-125 (¹²⁵I) binding approach was used to confirm the binding of caltrin to osteoblasts and osteoclasts. The presence of low background and the ability to quantify the interaction makes this technique a powerful tool in examining the binding of proteins to specific cell types. Recombinant caltrin protein (see Example 3) was labelled with radioisotope ¹²⁵I using the lactoperoxidase (Thorell & Johansson, 1971, Biochim Biophys Acta 251, 363-369). Briefly, 5 μg of recombinant caltrin protein was added to lactoperoxidase and incubated for 30 minutes. The labelled reaction was initiated by the addition of 1 mCi of Na¹²⁵ I and hydrogen peroxide. The reaction was halted using stop reaction consisting of KI, albumin and sodium azide. The labelled protein sample was then separated on a Sephadex G-50 column. Forty samples were eluted from the column and their activity measured in an autogamma counter. This iodination generated a standard labelling curve with the highest radioactivity in sample 20, corresponding to radiolabelled caltrin (¹²⁵I-caltrin), and the second highest activity in sample 28 (FIG. 9), corresponding to free iodine. The presence of ¹²⁵I-caltrin in the eluted samples was further confirmed by performing a 17.5% SDS-PAGE gel analysis and exposing the gel to a radiographic film.

Radiolabelled caltrin protein was subsequently used in a qualitative binding experiment. Briefly, calvarial osteoblasts, ST2, COS-7, chondrocytes, RAW_{246.7} cells and RAW_{246.7} cell-derived osteoclasts were seeded onto 12 well plates at a cell density of 2×10³ in 0.5 mL of culture media. The cells were incubated overnight in a humidified chamber at 37° C., 5% CO₂. Cells were subsequently washed and incubated with Blocking Buffer (serum-free growth media, 1% Fetal Bovine Serum and 50 mM HEPES), on ice for 10 minutes. Approximately 10 ng of labelled ¹²⁵I-caltrin, with or without a 75 fold excess of unlabelled caltrin, was added to the appropriate wells. Cells were incubated with caltrin for 2 hours at 4° C. Cells were then washed with cold PBS 5 times and solubilised with 0.2M NaOH and 1% SDS. The activity of each sample was then measured using an autogamma counter.

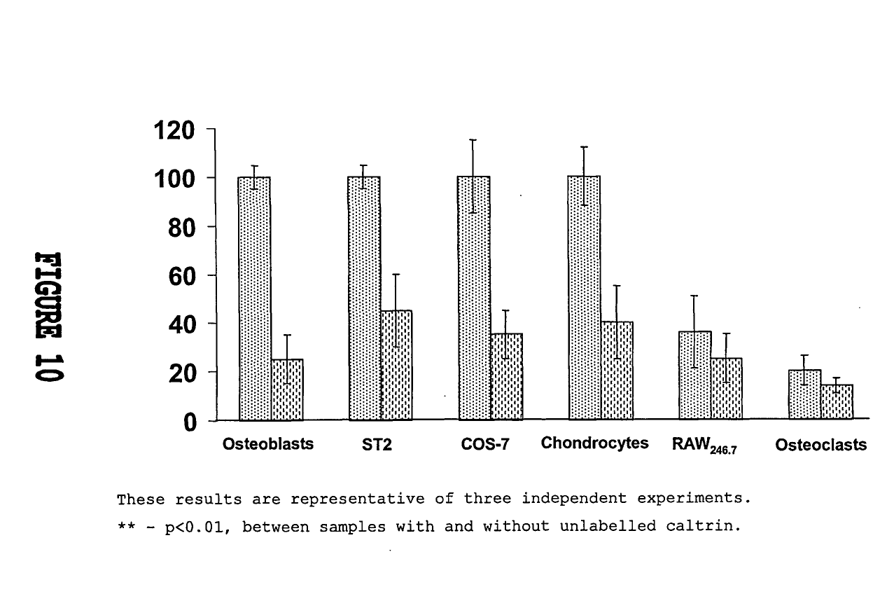

FIG. 10 illustrates a qualitative binding analysis of ¹²⁵I-caltrin to primary calvarial osteoblasts, ST2-osteoblast like cells, COS-7, human chondrocytes, RAW_{246.7} cells and RAW_{246.7} cell-derived osteoclasts, with competition by unlabelled recombinant caltrin. Evidently, caltrin was capable of binding to calvarial osteoblasts, ST2 osteoblast-like cells, COS-7 and human chondrocytes but did not bind to RAW_{246.7} cells or RAW₂₄₆ cell-derived osteoclasts. These observations demonstrate that caltrin binds to osteoblastic cells but does not bind to osteoclasts or their precursor cell lines. This is indicative of a paracrine mode of action where the osteoclast secretes a factor that binds to osteoblasts.

The interaction between osteoblasts and caltrin suggests that a paracrine mode of action exist for this osteoclast-derived factor. Further characterisation of this binding was performed in order to elucidate the binding affinity, the number of receptors and whether there exists single or multiple binding sites. The Scatchard Analysis, a mathematical technique of linearising data from binding experiments, was used to quantify the interaction between osteoblasts and caltrin.

Briefly, to produce the saturation binding curve necessary for Scatchard Analysis, increasing concentrations of ¹²⁵I-caltrin, 5, 10, 20, 40, 80 and 160 ng were added to 2×10⁵ primary mouse calvarial osteoblastic cells with or without excess unlabelled caltrin at 4° C. for 2 hours. The cells were subsequently washed, solubilised and their activity measured. The specific binding was calculated by subtracting the Non-Specific Binding (NSB) from the Total Binding counts. To produce a competition curve, calvarial osteoblastic cells were incubated with increasing concentrations of unlabelled recombinant caltrin in the presence of 10 ng of ¹²⁵I-caltrin for 2 hours at 4° C. The competition curve, saturation binding curve, Scatchard plot and subsequent calculations of Kda and binding sites per cell were produced using Graphpad Prism 4.0 software.

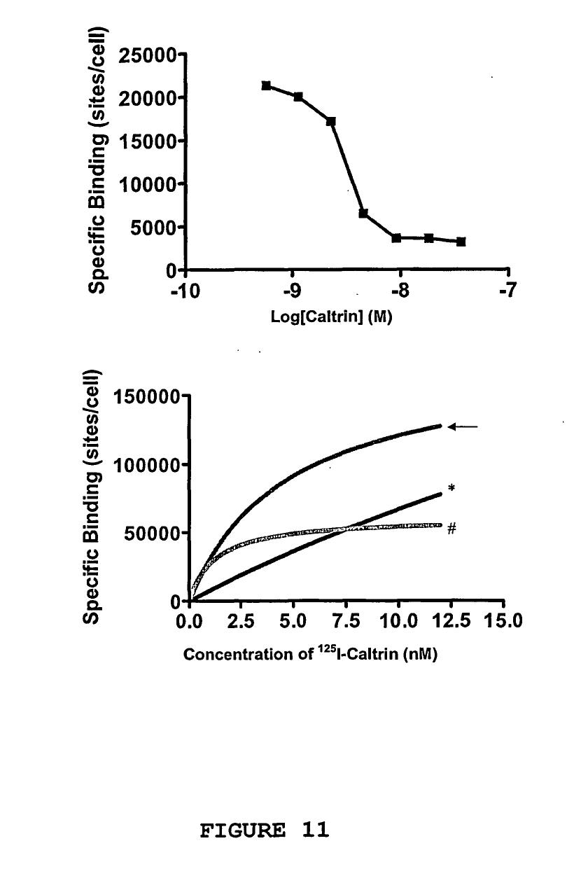

As can be observed in FIG. 11A, unlabelled caltrin competes for binding with labelled caltrin and increasing concentrations of the unlabelled protein replaces the labelled protein in a logarithmic pattern. The calculated binding affinity (Kd) from the competition curve is approximately 2.2 nM with 1.4×10⁴ binding sites per cell (Bmax), which is indicative of a strong interaction between caltrin and osteoblasts.

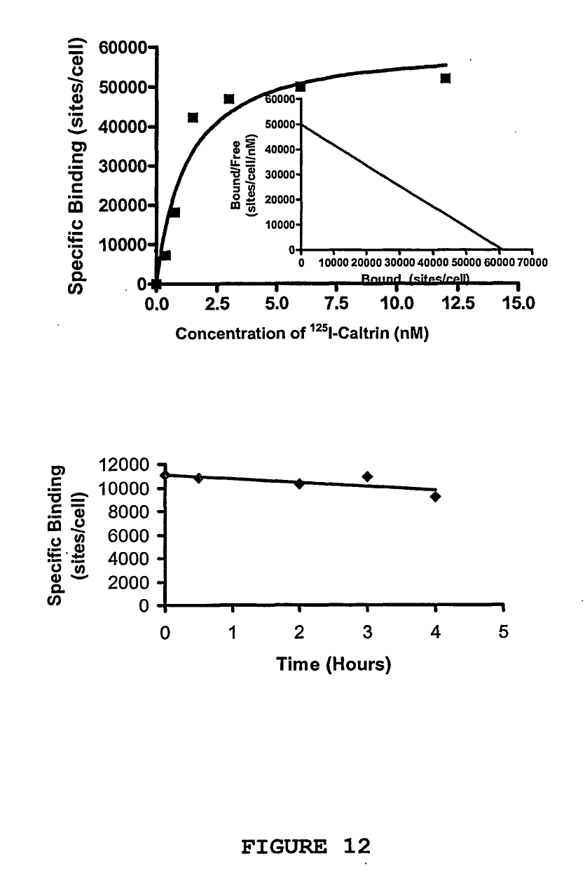

The final Scatchard experiment performed was a saturation-binding curve and its linearisation into the Scatchard Plot. This experiment involves four separate procedures. The first is the total binding, with increasing concentrations of ¹²⁵I-caltrin alone. The second is non-specific binding, with increasing concentrations of ¹²⁵I-caltrin in the presence of a constant amount of excess unlabelled caltrin. The third involves specific binding, which is merely the subtraction of non-specific binding from the total binding. Lastly, the specific binding curve is linearised into the Scatchard Plot. FIG. 11B illustrates a saturation binding curve between osteoblasts and caltrin with non-specific binding (blue/*), total binding (red/arrow) and specific binding (green/#) shown. FIG. 12A demonstrates the specific saturation binding curve with the Scatchard Plot shown in the inset. These two figures demonstrate that as the concentration of ¹²⁵I-caltrin added to osteoblastic cells is elevated, the number of occupied binding sites increases until the saturation point is reached, that is, all the binding sites are occupied. The calculated binding affinity is 1.2 nM with approximately 6×10⁴ binding sites per cell. These values further reinforce the presence of a strong interaction between osteoblasts and caltrin. Interestingly, the Scatchard plot shown (FIG. 12A) in the inset conforms to a normal one-site binding pattern, indicating that caltrin binds to one type of receptor on the cell.

To further quantify the binding between ¹²⁵I-caltrin and osteoblasts, a ligand-receptor dissociation assay was performed. This type of experiment can graphically demonstrate the strength of binding between caltrin and osteoblast cells, as it shows whether the bound labelled caltrin protein can be displaced by free unlabelled protein. Briefly, recombinant labelled ¹²⁵I-caltrin was incubated with osteoblasts for 1 hour, subsequently washed and incubated with excess unlabelled caltrin for the indicated time points on FIG. 12B. As illustrated, the binding between caltrin and osteoblasts is very strong, and poorly reversible, since a negligible decrease in the number of ¹²⁵I-caltrin bound sites was observed after cells were washed and incubated with excess unlabelled caltrin. Note that in this assay, it is the displacement of labelled caltrin by unlabelled caltrin that is being measured rather than competition of the two proteins.

### EXAMPLE 5

### Caltrin Receptor Studies

In order to elucidate the function of caltrin, or to use this protein therapeutically, we investigated possible receptor or interacting partners, utilising the Yeast Two-Hybrid system as described in Gietz et al. (1997) (Mol Cell Biochem 172, 67-79). Briefly, the caltrin gene as described above, was ligated into the DNA binding domain of the pBTM116 bait vector to create a pBTM116-caltrin bait vector. This was subsequently transformed into yeast cells. Ten millilitres of YPAD was inoculated with a single colony of L40 Yeast and cultured at 30° C., 225 RPM. The optical density (OD) of the yeast culture was constantly examined to observe the growth of cells. When the OD reached 0.4, the cells were further inoculated into 50 mLs of YPAD and cultured for an additional 3 hours. Cells were subsequently washed with 40 mLs of TE buffer (2500 RPM for 10 minutes) and resuspended in 2 mLs of 100 mM LiAc/0.5×TE. One hundred microlitres of yeast suspension was then added to an eppendorf tube containing 1 μg of pBMT116-caltrin bait vector, 100 μg of denatured sheared salmon DNA and 700 μL of 100 mM LiAc/40% PEG-3350/1×TE. The suspension was incubated at 30° C. for 30 minutes with shaking at 225 RPM. After incubation, 88 μL of DMSO was added to the suspension and subsequently heat shocked at 42° C. for 7 minutes. The cells were washed with 1 ml of TE and finally resuspended with 50 μL of TE. The suspension was plated onto agar with histidine (−UTL) to allow the growth of the L40-caltrin yeast cells.

Once the L40-caltrin yeast cells had grown, they were removed and co-transformed with the library prey plasmids. Five millilitre of Yc/−trp, −ura media was inoculated with a single L40-caltrin colony and cultured for 24 hours at 30° C., 225 RPM. The next day, the cells were added to 100 mL of additional Yc/−trp, −ura media and cultured for another 24 hours at 30° C., 225 RPM. The cell suspension was then added to 1 L of pre-warmed YPAD and grown for 3 hours at 30° C., 225 RPM or until the OD was 0.3. The cell suspension was then was washed twice. First with 500 mL of TE for 5 minutes at 5000 RPM and second with 100 mM LiAc/0.5×TE. The L40-caltrin yeast suspension was then transformed with denatured salmon sperm DNA and 500 μg of the mouse embryonic cDNA prey plasmid library. Additionally, 140 μL of 100 mM LiAc/40% PEG-3350/1×TE was added to the suspension and incubated at 30° C. for 30 minutes at 225 RPM. After incubation, 17.6 mLs of DMSO was added to the cell suspension and heat shocked at 42° C. in a water bath for 6 minutes. The transformed cells were subsequently added to 400 mL of YPA media and cooled to room temperature. The cells were then washed with 500 mLs of YPA, resuspended with 1 L of YPAD media and cultured for 1 hour at 30° C. with gentle shaking (150 RPM). After incubation, cells were washed and resuspended in 1 L of −UTL media. The cells were then grown for 16 hours at 30° C., 225 RPM. The next day, the cells were washed twice with −THULL and resuspended in 10 mL of −THULL media. Finally, 5, 10, 25, 50 and 100 μL of the −THULL suspension was aliquoted, spread onto plates with and without histidine (−UTL and −THULL) and grown at 30° C. for 3 days.

Approximately 50 positive colonies, with each representing one single protein-to-protein interaction, were removed and isolated and subsequently re-screened to confirm their interaction with caltrin. The secondary screening demonstrated that only 9 out of the 50 clones tested positive for growth on medium lacking histidine. Each positive cloned gene was then sequenced and translated into a peptide sequence by using the Institute of Virus Research Molecular Biology Translation tool (http://genzi.virus.kyoto-u.ac.jp). The gene and peptide sequence was then searched against a database of known genes and proteins (www.ncbi.nlm.nih.gov/BLAST/). Table 2 shows the identity of the 9 clones as shown by the nucleotide (BLASTN) and protein (BLASTP) database scan.

TABLE 2
SUMMARY OF THE 9 CLONES IDENTIFIED
FROM THE YEAST TWO-HYBRID

| Arbitrary | BLASTN Accession | BLASTP Accession | Proposed |
|---|---|---|---|
| Abbreviation | Number | Number | Function |
| NC1 | gi3851716 | No significant | Cloning |
| | pSOS cloning vector | match | Vector |
| | | | Unknown |
| | | | Peptide |
| NC5 | gi23346622 | gi21399982 | Modulating |
| | Translation | Hypothetical | protein |
| | Initiation Factor | Protein | translation |
| NC10 | gi4503526 | gi23271707 | Modulating |
| | Translation | Translation | protein |
| | Initiation Factor | Factor | translation |
| NC14 | gi20864619 | gi20879871 | Nucleosomal |
| | High Mobility Group | Nucleosomal | synthesis |
| | Protein | protein | |
| NC18 | gi27695343 | No significant | NADH |
| | Diaphorase 1 | match | Reductase |
| | | | Unknown |
| | | | Peptide |
| NC27 | gi191766 | gi191767 | Growth |
| | Acrogranin | Acrogranin | factor |
| NC33 | gi28488574 | gi8248421 | Modulating |
| | Ribosomal Protein | Kinesin | Protein |
| | 60S | | Translation |
| NC37 | gi28488574 | gi8248421 | Modulating |
| | Ribosomal Protein | Kinesin | Protein |
| | 60S | | Translation |
| NC46 | gi21434744 | Gi27687389 | Cell |
| | EMILIN | EMILIN | Migration |
| | | | and adhesion |

We predict that two out of the nine clones have the potential to be an interacting partner or receptor for caltrin. NC27 codes for acrogranin (accession number: gi191767), while NC46 codes for EMILIN (accession number: gi27687389). Both of these clones, unlike the rest, had 100% homology in the BLASTN and BLASTP search. A sweep of the current literature indicates that acrogranin, also known as progranulin or granulin-epithelin precursor, is a large 67 kDa protein that is expressed in a wide variety of cells (Baba et al., 1993, Mol Reprod Dev 34, 233-243; He and Bateman, 1999, Cancer Res 59, 3222-3229; Jones et al., 2003, Gynecol Oncol 88, S136-139).

Acrogranin has been implicated in cellular growth, proliferation, differentiation and even regulating cell survival. One study has shown that Acrogranin is localised on the head and body of spermatozoa and is involved in the acrosome reaction as well as spermatogenesis (Baba et al. supra). Likewise, caltrin has also been implicated in the acrosome reaction and has been demonstrated to bind to the head, mid-piece and tail of sperm cells.

EMILIN, also known as Elastin Microfibril Interface Located Protein, is a 115 kDa glycoprotein originally characterised on the surface of amorphous elastin (Colombatti et al., 2000, Matrix Biol 19, 289-301). EMILIN is involved in the cellular migration and adhesion of several cells, such as fibroblasts, haematopoietic and smooth muscle cells, to elastin, a glycoprotein matrix similar to collagen of bone (Colombatti et al. supra). Very recently one study has demonstrated that EMILIN-5, an isoform of the EMILIN family, is expressed in osteoblasts and may function in skeletal development (Doi et al., 2004, Biochem Biophys Res Commun 313, 888-893). It is possible that the binding between caltrin and EMILIN on osteoblasts may facilitate cellular migration and adhesion to the bone surface.

### EXAMPLE 6

### Caltrin Elevates Osteoblast Proliferation

Bioinformatic searches disclosed in Example 1 demonstrated that caltrin has similar domains to EGF, a potent growth factor that is involved in osteoblastic proliferation. To test the hypothesis that caltrin induces the growth and proliferation of osteoblasts, the alamar blue proliferation assay was used. This methodology detects metabolic cellular growth by measuring the reduction of alamar blue in the mitochondria (Moursi et al., 2002, Cleft Palate-Craniofacial Journal 39, 487-496). As the cells proliferate the dye is further reduced and the fluorescent intensity of the alamar dye is measured and quantified.

Briefly, osteoblastic cells (primary mouse calvarial and human MG63) were seeded onto 96 well plates at a density of 2×10³. The appropriate concentration of caltrin was added to the wells and incubated for 24 and 48 hours with and without serum. Ten microlitres of alamar blue reagent was added to each 100 μL well, incubated for 1 hour and analysed using the FluoStar Optima microplate reader (BMG Lab Technologies, Offenburg, Germany) at an excitation wavelength of 544 nM and emission wavelength of 590 nM.

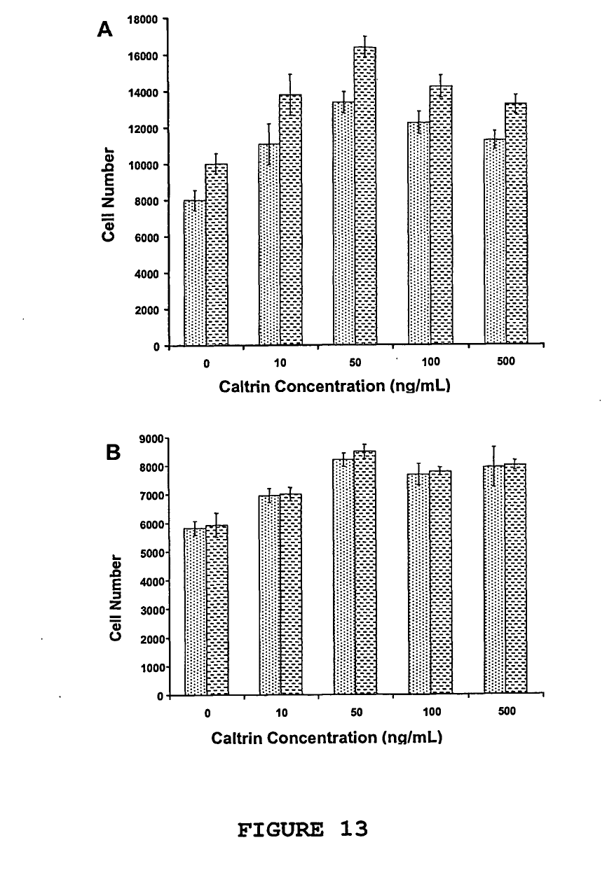

FIG. 13A shows that recombinant caltrin significantly elevates the growth and proliferation of primary calvarial osteoblastic cells at 24 and 48 hours stimulation in normal serum media compared to untreated controls. Osteoblastic cell number increased in a dose dependent manner with 50 ng/mL of caltrin protein eliciting the most significant increase (approximately 70%). When primary calvarial osteoblasts were cultured in serum-free media and exposed to caltrin, an increase in proliferation was also observed (FIG. 13B). In 24 hours there was a 35% increase in cell number (50 ng/mL) compared to untreated controls. Furthermore, the growth of osteoblasts in normal media is higher compared to that in serum free media. This is most likely due to the synergistic effect of the fetal calf serum in the medium. Interestingly, proliferation at 48 hours serum-free did not significantly augment compared to cells at 24 hours.

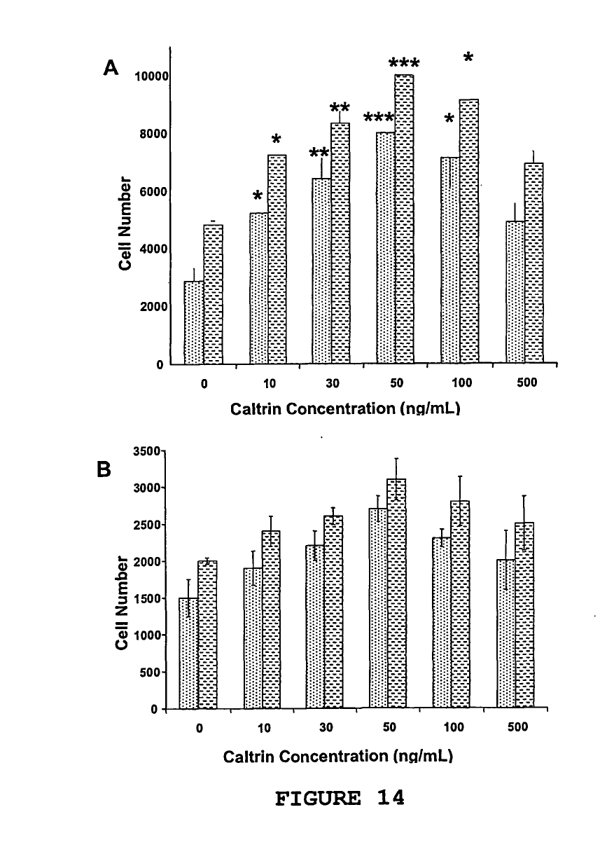

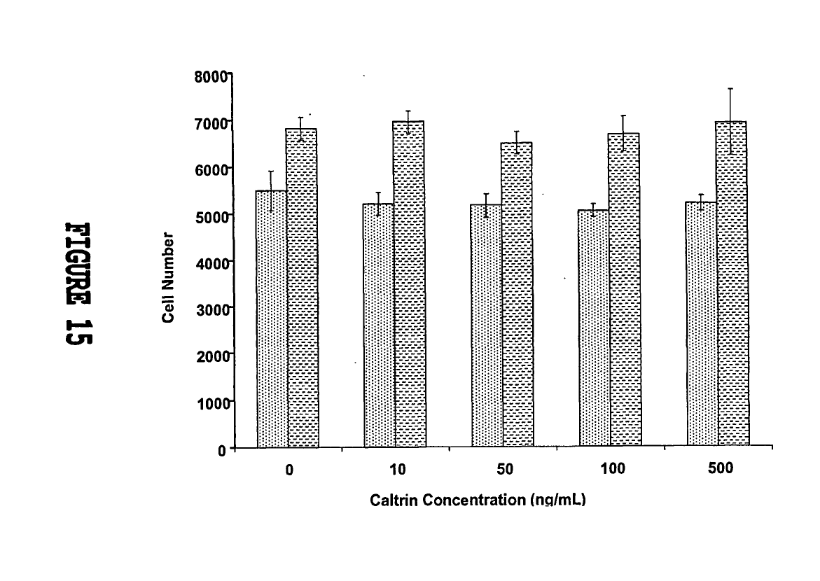

Several other cell lines were examined to determine whether caltrin had a proliferative effect. FIG. 14A reveals significant proliferation of MG-63 human osteoblastic cell line in normal media when exposed to caltrin for 24 and 48 hours. Interestingly the proliferation is considerably elevated compared to primary calvarial osteoblasts, with the most significant effect corresponding to 50 ng/mL (approximately 200% increase). When MG-63 cells are cultured in serum-free media (FIG. 14B) and exposed to caltrin, a substantial increase in cell number is observed, but is relatively small compared to cells in normal medium. Similarly to primary osteoblasts, MG-63 cell lines cultured in 48 hours serum-free do not significantly proliferate compared to cells in 24 hours serum-free. When RAW_{246.7} cells were exposed to caltrin in normal medium for 24 and 48 hours, no proliferation was observed (FIG. 15). This is in agreement with the binding results discussed in Example 4 suggesting that caltrin has no affect on osteoclastic precursors.

Another interesting observation is the lack of a strong stimulative effect of caltrin at higher concentrations. For primary calvarial osteoblasts in both normal and serum-free media, the maximal proliferation occurs at 50 ng/mL, but has no significant proliferative effect from 100 ng/mL to 500 ng/mL. This is further observed in MG-63 human osteoblast-like cells, with 500 ng/mL of caltrin having cell numbers near control level. Despite this, however, the results demonstrate that caltrin increases the growth and proliferation of osteoblastic cells at a dose dependent manner. Thus, identifying the first function of caltrin in bone.

### EXAMPLE 7

### Caltrin Increases Bone Mineralisation

The exclusive function of osteoblasts is to form osseous tissue identifiable as bone. This involves the synthesis and deposition of hydroxyapatite calcium crystals in the bone matrix. To determine whether caltrin can induce or facilitate osteoblast bone formation in vitro, a bone mineralisation experiment was executed. This assay measures the amount of calcium deposition, which is synthesised by mature osteoblasts, within the confluent cell layer. The synthesised calcium crystals are mobilised within localised areas on the layer of osteoblasts and are known as “nodules”. The longer the osteoblasts are allowed to deposit and mineralise these crystals, the higher the amount of nodules that appear. Furthermore, these nodules can be stained and quantified and can thus be correlated with bone formation in vitro.

Briefly, primary calvarial osteoblastic cells were seeded onto 6 well plates and grown until confluent. Cell medium was then exchanged with differentiation media (100 g/mL Ascorbic Acid and 10 mM β-glycerophosphate) and the appropriate concentration of caltrin or dexamethasone (positive control) was added to each well. A single well was left with normal media and untreated with caltrin (negative control). Fresh differentiation medium, caltrin protein and dexamethasone was replaced every second day for 21 days. To visualise mineralisation and calcium nodule formation, the cells were stained with Von Kossa (Schenk et al., 1984, In Preparation of calcified tissues for light microscopy, G. R. Dickson, ed. (Amsterdam, Elsevier). Briefly, cells were fixed with 4% Paraformaldehyde (made in TBS). Cell medium was removed and washed three times with ddH₂0. Approximately 500 μL of Silver Nitrate was added to each well in the dark for 15 minutes and subsequently exposed to UV light for 5 minutes. Cells were then treated with 5% sodium thiosulfate or Farmers Reducer for 5 minutes. Each well was then washed with ddH₂O and air dried. Quantification was done by scanning densitometry using the program ImageJ (http://rsb.info.nih.gov/ij/).

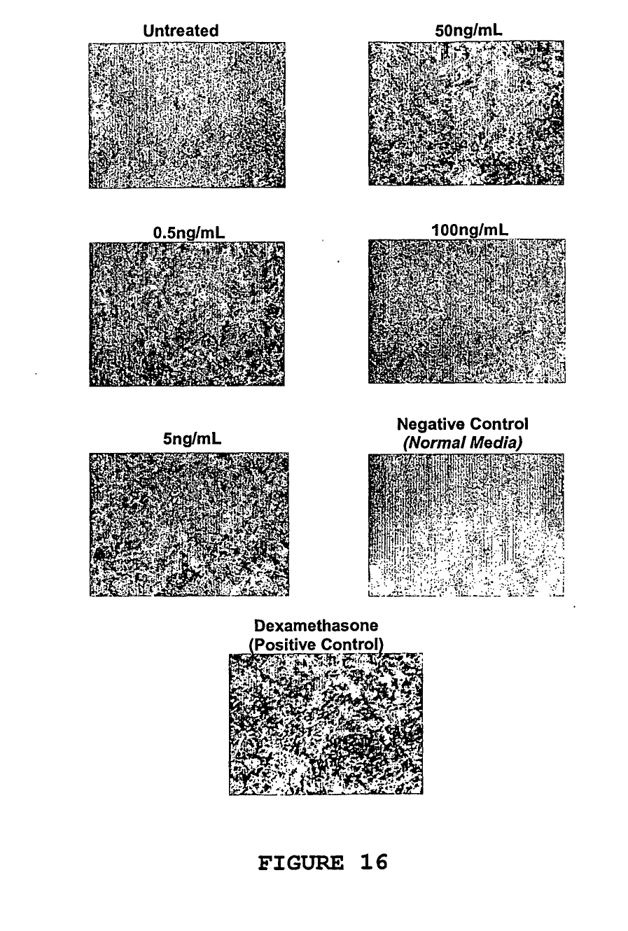

FIG. 16 provides a qualitative assessment of osteoblast mineralisation stained with the von Kossa procedure. Osteoblastic cells in the presence of caltrin exhibit significantly more mineralisation nodules (brown), compared to cells in the absence of the protein (untreated). Likewise, the positive control with dexamethasone also produced significantly higher numbers of nodules. Moreover, as expected calvarial osteoblasts without differentiation media (negative control) did not induce mineralisation. Interestingly, when osteoblastic cells are exposed to caltrin at a concentration of 100 ng/mL the amount of bone nodules drastically decreases, producing a result similar to untreated osteoblasts. Osteoblastic cells were quantified using scanning densitometry.

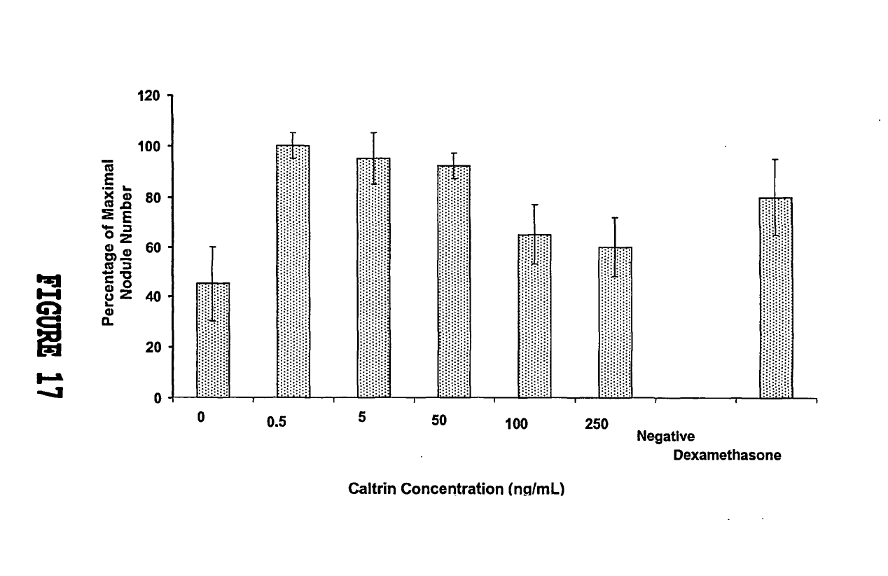

FIG. 17 illustrates a quantitative assessment of von kossa stained osteoblast mineralisation. In the presence of 0.5 ng/mL of caltrin there is a significant increase in nodule numbers (around 100%) compared to untreated controls. The same increase is observed for the dexamethasone control. Interestingly, the amount of mineralised nodules between 0.5 ng/mL to 50 ng/mL of caltrin remains invariable. However, when cells are treated with 100 ng/mL and 250 ng/mL of caltrin the amount of nodules does not significantly increase. In fact the mineralisation is similar to that of the untreated controls. This may indicate an inhibition of mineralisation nodules at high caltrin concentrations.

This experiment illustrates that caltrin can promote the deposition and synthesis of calcium crystals in mineralisation nodules, an indicator of bone formation. Furthermore, the results demonstrate a specific bone-related function for caltrin.

### EXAMPLE 8

### Osteoclastogenesis

To ascertain the effect of caltrin on osteoclasts, the osteoclastogenesis facet was examined. Briefly, RAW_{246.7} cells and Spleen cells were cultured onto 96 well plates and treated with either RANKL alone or 50, 100, 300 and 500 ng/mL of caltrin, alone. In addition, RANKL (30 ng/mL) and caltrin (50 ng/mL) were added together to ascertain whether the two proteins had a synergistic effect on each other. Negative controls involved the addition of PBS alone. The cells were cultured for five days with the medium being changed every second day. M-CSF was further added to the spleen cells to facilitate the generation of osteoclasts.

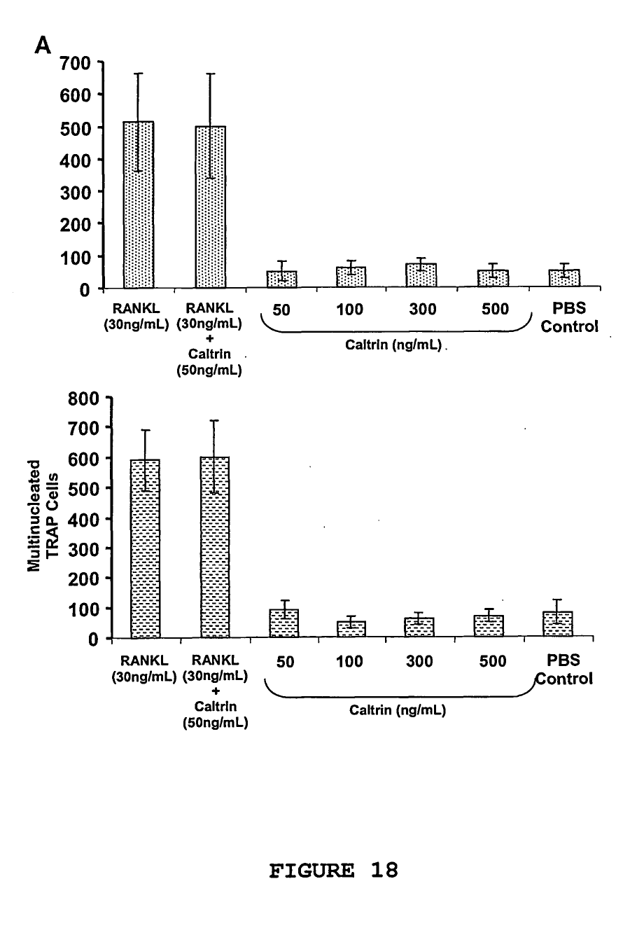

FIG. 18A illustrates a quantitative assessment of the TRAP-stained osteoclastogenesis experiment. The addition of caltrin to precursor cells does not promote or inhibit the formation of osteoclasts. In fact, cells treated with a mixture of caltrin and RANKL have approximately the same number of osteoclasts as cells treated with RANKL alone.

Spleen culture-derived osteoclasts were further examined. Like RAW_{246.7} cells, spleen culture were seeded onto 96 well plates and treated with RANKL and mCSF for 7 days. In addition, the spleen cells were treated with caltrin alone and combinations of the RANKL-MCSF mixture with caltrin. As demonstrated in FIG. 18B, caltrin has negligible affect on the differentiation of spleen-cell derived osteoclasts. Together, these observations indicate that caltrin neither activates nor inhibits the formation of osteoclasts in vitro.

### EXAMPLE 9

### Osteoclast Survival

To ascertain whether caltrin can promote the continuation of osteoclasts in vitro, a survival assay was performed. RAW_{246.7} cells were cultured onto 96 well plates with the addition of RANKL. Briefly, RAW_{246.7} cells were prepared by the method of Xu et al. (2000) (Journal of Bone & Mineral Research 15, 2178-2186). Once RAW_{246.7} cell-derived osteoclasts were formed on day 5 of differentiation, the cells were washed and exchanged with medium containing either RANKL (positive control), 50, 100 or 500 ng/mL of caltrin. Negative controls were further prepared by adding PBS vehicle to the cells. Osteoclasts were cultured for an additional 24 hours and subsequently stained for TRAP, as described preciously (Xu et al. supra).

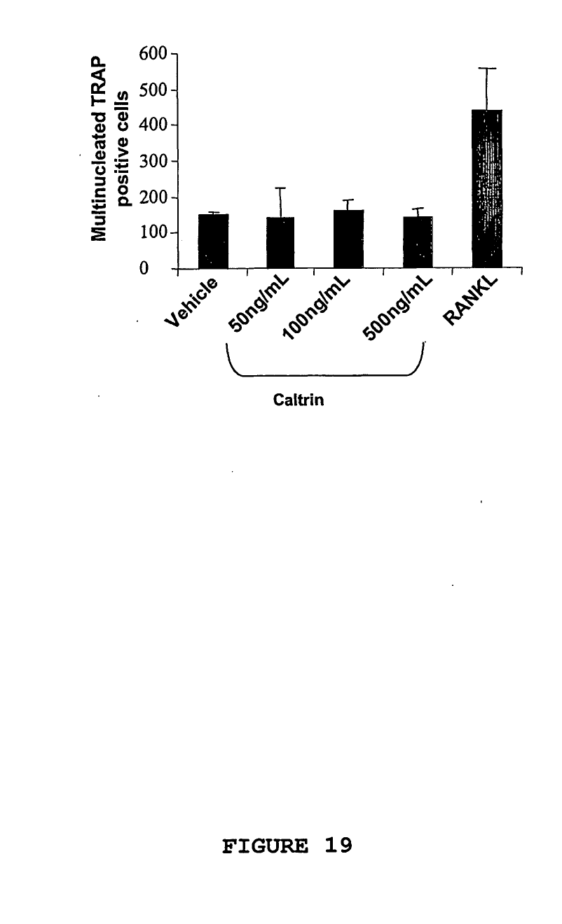

It was observed that in the presence of caltrin, no change in osteoclast number was obtained. The only effect observed was that cells in the presence of RANKL, had significantly higher TRAP stained cells compared to those with caltrin or vehicle control. FIG. 19 is a graphical representation of the number of TRAP-stained osteoclasts. As can be seen, no significant increase in osteoclast number was observed compared to vehicle control. Altogether the results demonstrate that caltrin does not promote the survival of osteoclasts, and it reinforces again that caltrin does not function on osteoclastic cells.

### EXAMPLE 10

### Osteoclastic Bone Resorption

To fully examine whether caltrin can affect osteoclasts, a bone resorption pit assay was performed. Briefly, dentine bone slices were added to empty 96 well plates and sterilised overnight by ultraviolet light exposure. Primary osteoclasts isolated from one-day old rats were added to each well and incubated for 60 minutes. The bone culture was washed to remove less adherent cells and then treated with 50, 100 and 300 ng/mL of caltrin. The cells were incubated in a humidified chamber at 37° C. (5% CO₂) in culture medium for 24 hours. The bone slices were subsequently examined under a scanning electron microscope for evidence of bone excavations, indicative of bone resorption pits.

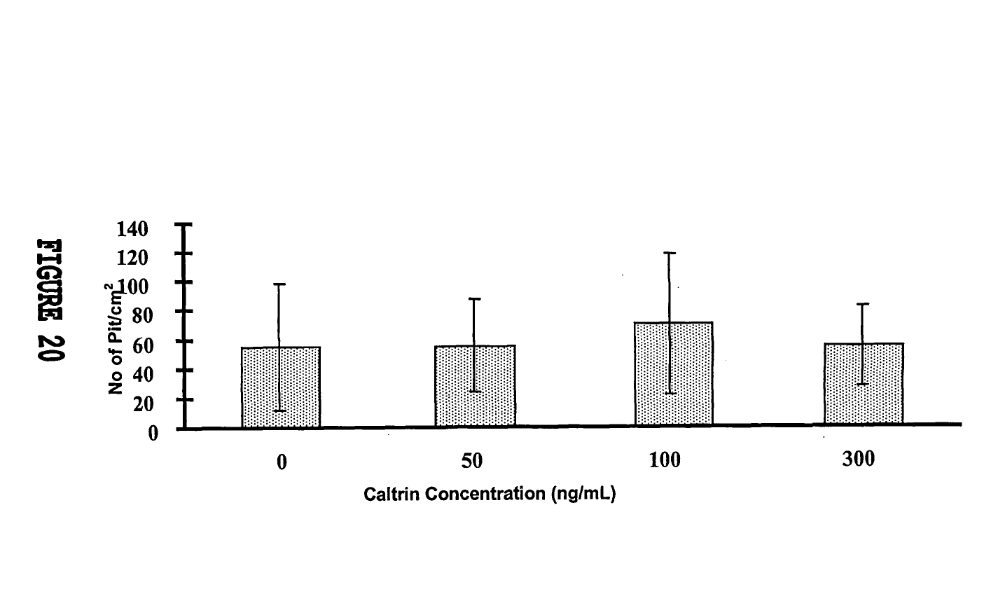

FIG. 20 demonstrates a quantitative assessment of the number of bone resorption pits produced by bone marrow-derived osteoclasts. The number of bone resorption pits did not significantly alter in the presence of caltrin for both types of osteoclastic cells, indicating that the protein has no effect on osteoclastic function.

### EXAMPLE 11

### Caltrin Elevates Intracellular Calcium via Internal Calcium Store Depletion

Intracellular calcium was monitored utilising the BMG FluoStar Optima, a microplate reader that has been used extensively in calcium measurements (Heding et al., 2002, J. Recept & Sig, Transduct Res., 22, 241-252). Briefly, COS-7, ST2 and primary calvarial osteoblasts were seeded onto special black glass 96 well plates and grown overnight in a humidified incubator at 37° C. and 5% CO₂. Cells were then loaded with Fluo-3AM (10 uM) for 40 minutes prior to addition of caltrin. Fluo-3 was measured using an excitation filter of 485 nm and emission filter of 520 nm. Emission was measured from the bottom of the wells, while fluorescence was excited from the top.

To ascertain the mode of intracellular calcium elevation the following experiments were performed. Firstly, the internal calcium store was depleted by addition of thapsigargin. Calvarial osteoblasts were loaded with Fluo-3AM and then treated with 2 μM of thapsigargin. Once calcium was depleted, caltrin was subsequently added. Secondly, external calcium was removed by addition of EGTA. Calvarial osteoblastic cells were treated with 3 mM of EGTA to chelate calcium in the culture media. Fluo-3AM was subsequently loaded and cells were treated with caltrin. Thirdly., the Phospholipase C (PLC) pathway was examined. Calvarial osteoblasts were pre-treated with a PLC-inhibitor, U73122, for 10 minutes. Fluo-3AM was then loaded onto the cells and caltrin was added as per usual.

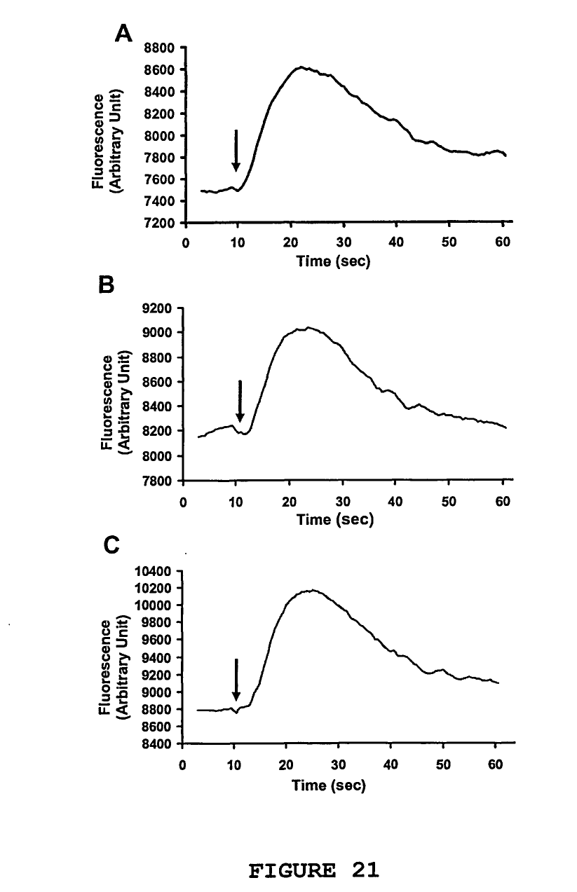

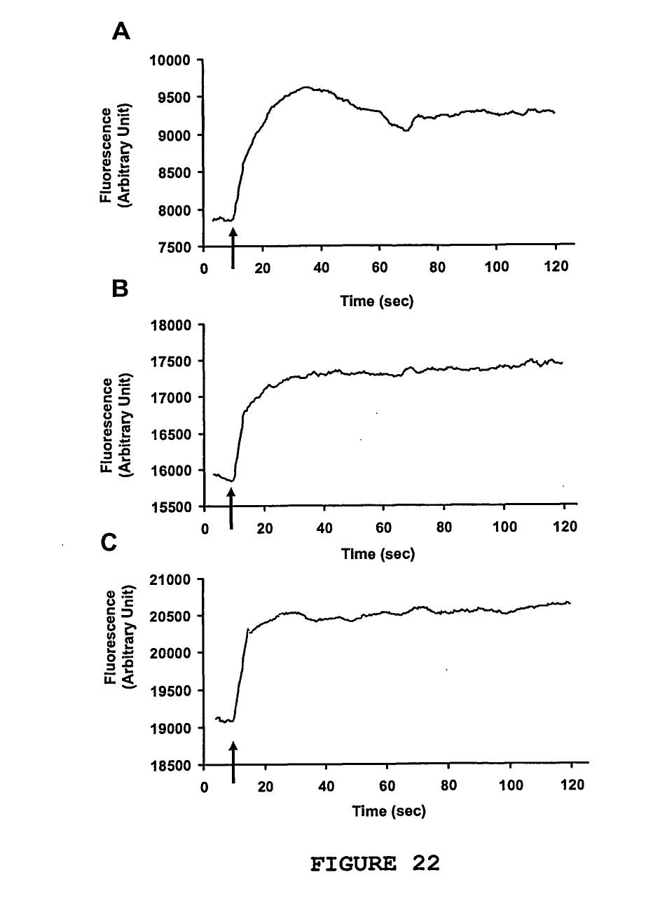

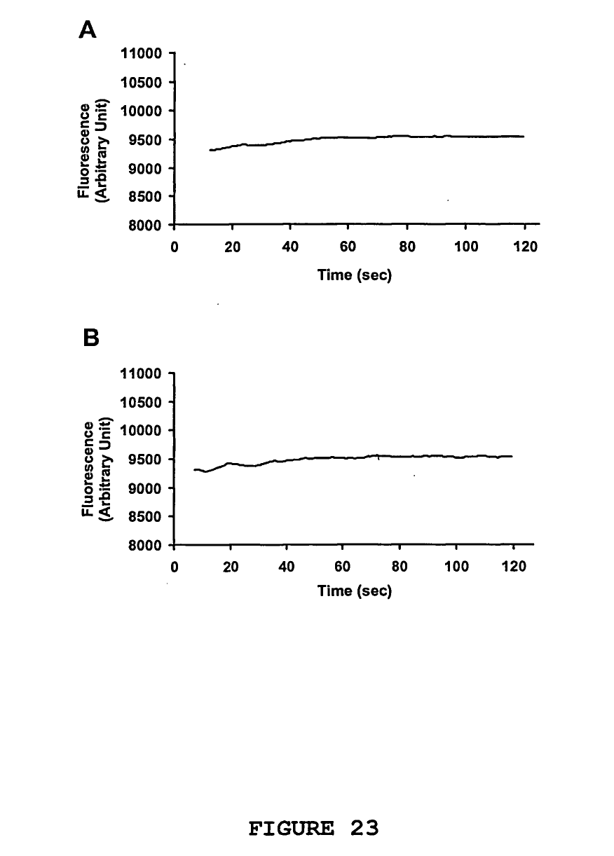

FIG. 21A demonstrates alterations in [Ca²⁺]ᵢ when 50 ng/mL of caltrin is exposed to primary calvarial osteoblastic cells. A rapid rise in [Ca²⁺]ᵢ is observed, which reaches a peak and gradually declines to basal level. Interestingly, when the same protein was exposed to ST2-osteoblast like cells (FIG. 21B) and COS-7 monkey kidney cells (FIG. 21C), the same elevation in [Ca²⁺]ᵢ was observed. These results coincide and reinforce the binding experiment (Example 4), which revealed that caltrin binds to primary calvarial osteoblast cells, as well as ST2 and COS-7 cells. When primary osteoblasts (FIG. 22A), ST2 (FIG. 22B) and COS-7 (FIG. 22C) cells were exposed to 500 ng/mL of caltrin, a slightly different calcium curve was revealed. A rapid rise was still evident for all three cell lines, but no gradual decline in [Ca²⁺]ᵢ was observed. In fact, the calcium level plateau and remained in its elevated state. This altered calcium pattern may represent an activation of a different osteoblastic process, or a toxicity effect. In contrast, RAW_{246.7} cell-derived osteoclasts (FIG. 23A) and RAW_{246.7} cells (FIG. 23B) did not respond to caltrin at any concentration. Likewise, this reinforces the binding results revealed in Example 4, which indicates that caltrin does not bind to RAW_{246.7} cell-derived osteoclasts or its precursor cell, RAW_{246.7}.

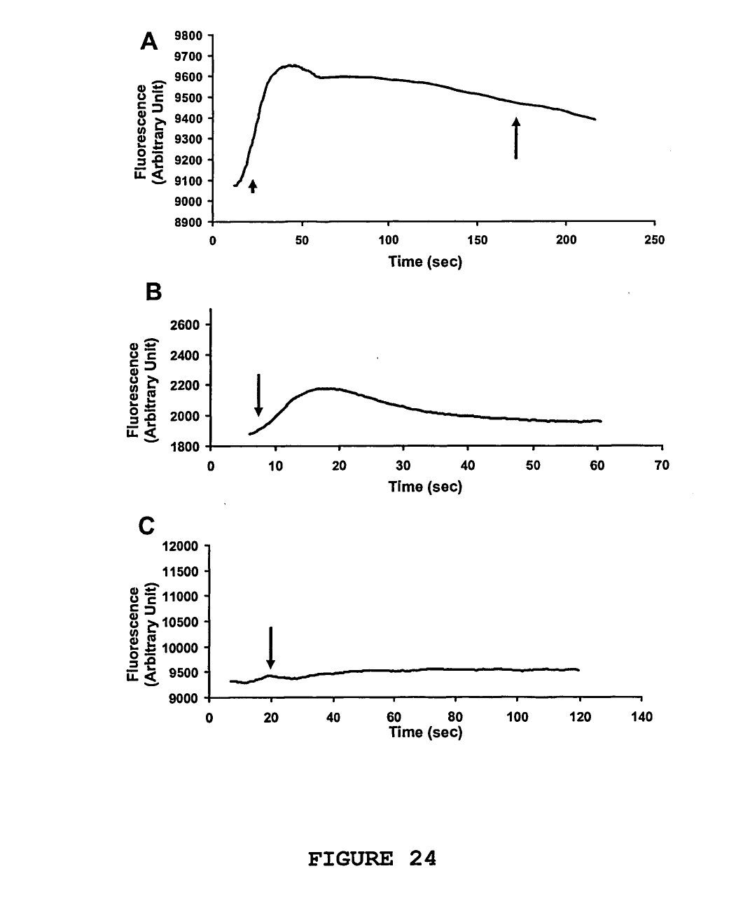

The BMG FluoStar Optima was further utilised to ascertain the mode of [Ca²⁺]ᵢ elevation; that is, whether caltrin elevates intracellular calcium via external calcium influx, or internal calcium store depletion. Three key experiments were designed. The first involved the application of thapsigargin, a drug that binds to the ATPase channel on the endoplasmic reticulum (ER). Thapsigargin effectively depletes the calcium store within the ER and further prevents the mobilisation of calcium from the cytosol into the organelle. In FIG. 24A, thapsigargin is added to primary calvarial osteoblastic cells at the 10-second time point. This induces a rapid rise in [Ca²⁺]ᵢ, which plateaus and begins declining to basal level. At the 170 second mark thapsigargin has depleted all the calcium within the endoplasmic reticulum store and addition of caltrin at this time does not elevate [Ca²⁺]ᵢ. The mode of [Ca²⁺]ᵢ increase was further examined by seeding osteoblasts in the presence of EGTA and subsequently adding caltrin (arrow, FIG. 24B). A rise in [Ca²⁺]ᵢ is observed in osteoblastic cells even when the external environment is completely devoid of calcium. To further ascertain whether caltrin is depleting internal calcium store or inducing an influx from the external environment, a Phospholipase C (PLC) inhibitor was utilised. The PLC-IP3 pathway initiates calcium store depletion in most cells, including osteoblasts. Inhibition of this pathway prevents the internal calcium store in the cells from being released. Osteoblastic cells were treated with U73122 (PLC inhibitor) for 10 minutes and subsequently loaded with Fluo-3AM and exposed to caltrin.

In FIG. 24C, U73122-treated osteoblasts do not respond to caltrin stimulation (arrow). This indicates that caltrin elevates [Ca²⁺]ᵢ via the PLC-IP3 internal calcium store depletion pathway

### EXAMPLE 12

### PI3K-Akt and ERK Signal Transduction Pathways

The phosphatidylinosital-3-kinase-Akt pathway has been established as a major mechanism behind cellular growth, differentiation and survival (Steelman et al., 2004, Leukemia 18, 189-218). Furthermore, an elevation in [Ca²⁺]ᵢ has been associated with PI3K-Akt activation (Danciu et al., 2003, FEBS Letters 536, 193-197). Thus it was logical to ascertain whether caltrin can activate these signalling cascades.

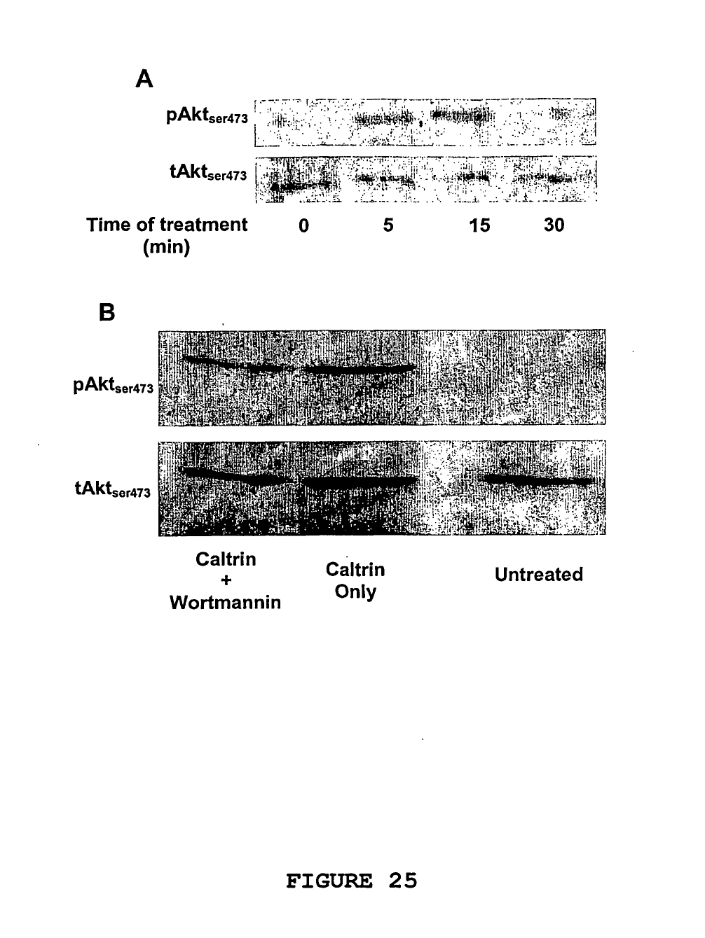

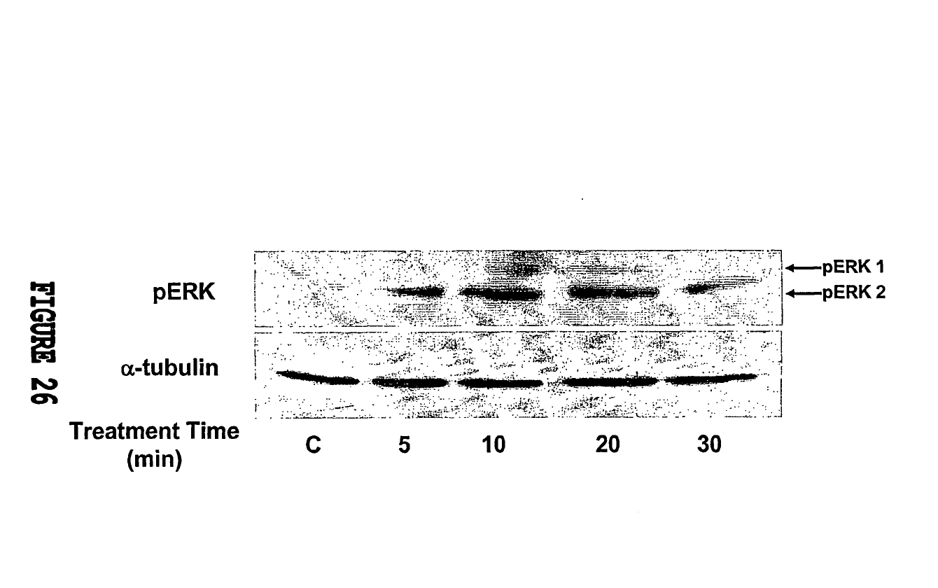

To detect Akt, quiescent primary calvarial osteoblasts were treated with caltrin and the cell lysate was subsequently extracted and detected using anti-Akt_{Ser473} antibody. In FIG. 25A Akt phosphorylation is transiently elevated in a time dependent manner with 5 to 15 minutes producing a maximal response. Interestingly, there was a significant decrease in Akt activation between 15 and 30 minutes, with the latter producing no Akt response. To ascertain whether the activation of PI3K is a pre-requisite for Akt phosphorylation a PI3K inhibitor, wortmannin, was utilised. In FIG. 25B, wortmannin partially attenuates caltrin-induced activation of Akt, suggesting the existence of other signal pathways that may be involved in the activation of PI3-Akt. To ascertain whether caltrin activates ERK, primary calvarial osteoblasts were treated with 50 ng/mL of caltrin at various time points. The cell lysate was extracted and detected using anti-ERK½ antibody. As evident in FIG. 26, caltrin transiently activates the ERK pathway with maximal stimulation at 10-20 minutes. Interestingly, only the ERK1 kinase was stimulated, which is in contrast with most reports that demonstrate activation of both ERKs using anti-ERK½ antibody.

### EXAMPLE 13

### Runx-Osterix Pathway

To ascertain whether caltrin can stimulate the gene expression of Runx and Osterix, RT-PCR was performed on calvarial osteoblastic cells. Briefly, primary calvarial osteoblasts were seeded onto 6 well plates and cultured for 15 days in the presence of 50 ng/mL caltrin and normal serum medium. Semi-quantitative RT-PCR was performed on the RNA extracted from these cells. It was observed that caltrin does not elevate the expression of Runx at any time point. Additionally, an absence in the gene upregulation of the Osterix transcription factor can also be observed. Interestingly, primary calvarial osteoblasts express basal level of Runx and Osterix (control) reinforcing that these cells are proper osteoblasts.

### EXAMPLE 14

### In Vivo Treatment Using Caltrin

In order to validate the treatment of bone disorders with caltrin, 50 Wistar rats of parity 5 can be randomised into groups of 5 animals. The groups can be treated for 2 or 4 weeks with placebo or with 50, 100, 200, or 400 μg/kg caltrin. All treatments can be given by subcutaneous injection every second day. Placebo-treated rats receive phosphate buffer saline (PBS). For qualitative assessment of bone formation, all the rats are given 10 mg/ml of calcein by intraperitoneal injection 5 days before treatment and tetracycline HCl, 15 mg/ml by intraperitoneal route 5 days before sacrifice.

The quantitative assessment of bone formation is determined by giving all the rats 30 pCi doses of [³H] tetracycline by subcutaneous injection on days 6, 5, and 4 before sacrifice. The vertebrae, left tibiae, and femora are removed and fixed in paraformaldehyde. The right tibiae is dehydrated under vacuum and ³H is extracted. Total ³H tetracycline activities are measured. The vertebrae, left tibiae, and femora are sectioned and subjected to bone histology examination as well as bone histomorphometric analysis. It is anticipated that the effects of caltrin on bone growth can be readily determined under physiological conditions. It can be seen that caltrin can enhance bone formation in vivo.

If the systemic effects of caltrin on the induction of bone formation is difficult to observe, then similar experiments can be performed, but using local administration of caltrin to femurs of mice using methods previously described.

1. A bone remodelling agent comprising a pharmaceutical composition comprising a therapeutically- or prophylactically-effective amount of caltrin or functionally active peptide fragment thereof together with a pharmaceutically acceptable carrier, wherein said caltrin is coded for by a nucleotide sequence comprising the nucleotide sequence shown in FIG. 1 (SEQ ID NO: 1) or FIG. 2 (SEQ ID NO: 2).

2. An agent according to claim 1, wherein said peptide fragment is about 10 to about 20 amino acids in length and having at least one biological activity of caltrin.

3. An agent according to claim 1, wherein said peptide fragment is about 10 to about 60 amino acids in length and having at least one biological activity of caltrin.

4. An agent according to claim 1, wherein said peptide fragment is about 10 to about 76 amino acids in length and having at least one biological activity of caltrin.

5. An agent according to claim 1, wherein said peptide fragment consists essentially of the amino acid sequence shown in FIG. 3 (SEQ ID NO: 3) or FIG. 4 (SEQ ID NO: 4).

6. An agent according to claim 1, wherein said peptide has the ability to elevate cytosolic calcium levels in osteoblasts and osteoblast-like cells.

7. An agent according to claim 1, wherein said peptide has the ability to induce bone formation.

8. An agent according to claim 1, wherein said peptide has the ability to elevate cytosolic calcium levels.

9. A method of treating or preventing bone disorders or diseases comprising the step of administering a pharmaceutical composition comprising caltrin or functionally active fragment thereof to an individual in need thereof.

10. A method according to claim 9, wherein the caltrin has an amino acid sequence as shown in FIG. 3 (SEQ ID NO: 3) or FIG. 4 (SEQ ID NO: 4), or cleaved product thereof having at least one biological activity of caltrin.

11. A method according to claim 10, wherein said cleaved product is about 10 to about 20 amino acids in length and having at least one biological activity of caltrin.

12. A method according to claim 10, wherein said cleaved product is about 10 to about 60 amino acids in length and having at least one biological activity of caltrin.

13. A method according to claim 10, wherein said cleaved product is about 10 to about 76 amino acids in length and having at least one biological activity of caltrin.

14. A method according to claim 9, wherein said caltrin or cleaved product thereof has the ability to elevate cytosolic calcium levels in osteoblasts and osteoblast-like cells.

15. A method according to claim 9, wherein said caltrin or cleaved product thereof has the ability to induce bone formation.

16. A method according to claim 9, wherein said caltrin or cleaved product thereof has the ability to elevate cytosolic calcium levels.

17. A method for inducing bone formation comprising the step of administering a pharmaceutical composition comprising caltrin or functionally active fragment thereof to an individual in need thereof.

18. A method according to claim 17, wherein the caltrin has an amino acid sequence as shown in FIG. 3 (SEQ ID NO: 3) or FIG. 4 (SEQ ID NO: 4), or cleaved product thereof having at least one biological activity of caltrin.

19. A method according to claim 17, wherein said cleaved product is about 10 to about 20 amino acids in length and having at least one biological activity of caltrin.

20. A method according to claim 17, wherein said cleaved product is about 10 to about 60 amino acids in length and having at least one biological activity of caltrin.

21. A method according to claim 17, wherein said cleaved product is about 10 to about 76 amino acids in length and having at least one biological activity of caltrin.

22. A method according to claim 17, wherein said caltrin or cleaved product thereof has the ability to elevate cytosolic calcium levels in osteoblasts and osteoblast-like cells.

23. A method according to claim 17, wherein said caltrin or cleaved product thereof has the ability to induce bone formation.

24. A method according to claim 17, wherein said caltrin or cleaved product thereof has the ability to elevate cytosolic calcium levels.

25. A method for forming bone matrix comprising the step of administering a pharmaceutical composition comprising caltrin or functionally active fragment thereof to an individual in need thereof.

26. A method according to claim 25, wherein the caltrin has an amino acid sequence as shown in FIG. 3 (SEQ ID NO: 3) or FIG. 4 (SEQ ID NO: 4), or cleaved product thereof having at least one biological activity of caltrin.

27. A method according to claim 25, wherein said cleaved product is about 10 to about 20 amino acids in length and having at least one biological activity of caltrin.

28. A method according to claim 25, wherein said cleaved product is about 10 to about 60 amino acids in length and having at least one biological activity of caltrin.

29. A method according to claim 25, wherein said cleaved product is about 10 to about 76 amino acids in length and having at least one biological activity of caltrin.

30. A method according to claim 25, wherein said caltrin or cleaved product thereof has the ability to elevate cytosolic calcium levels in osteoblasts and osteoblast-like cells.

31. A method according to claim 25, wherein said caltrin or cleaved product thereof has the ability to induce bone formation.

32. A method according to claim 25, wherein said caltrin or cleaved product thereof has the ability to elevate cytosolic calcium levels.

33. A method for increasing proliferation of osteoblasts comprising the step of administering a pharmaceutical composition comprising caltrin or functionally active fragment thereof to an individual in need thereof.

34. A method according to claim 33, wherein the caltrin has an amino acid sequence as shown in FIG. 3 (SEQ ID NO: 3) or FIG. 4 (SEQ ID NO: 4), or cleaved product thereof having at least one biological activity of caltrin.

35. A method according to claim 33, wherein said cleaved product is about 10 to about 20 amino acids in length and having at least one biological activity of caltrin.

36. A method according to claim 33, wherein said cleaved product is about 10 to about 60 amino acids in length and having at least one biological activity of caltrin.

37. A method according to claim 33, wherein said cleaved product is about 10 to about 76 amino acids in length and having at least one biological activity of caltrin.

38. A method according to claim 33, wherein said caltrin or cleaved product thereof has the ability to elevate cytosolic calcium levels in osteoblasts and osteoblast-like cells.

39. A method according to claim 33, wherein said caltrin or cleaved product thereof has the ability to induce bone formation.

40. A method according to claim 33, wherein said caltrin or cleaved product thereof has the ability to elevate cytosolic calcium levels.

41. A method according to claim 9, wherein the disease or disorder is selected from the group consisting of osteoporosis (including post menopausal osteoporosis, male and female senile osteoporosis and corticosteroid induced osteoporosis), osteoarthritis, Paget's disease, osteomalacia, prolonged bed rest, chronic disuse of a limb, anorexia, microgravity, exogenous and endogenous gonadal insufficiency, bone fracture, non-union, defect, prosthesis implantation, malignancy-related bone loss, and the like.

42. A method according to claim 9, wherein said individual is a mammal.

43. A method according to claim 42, wherein said mammal is a human subject.

44. A method of treating osteopenia, comprising administering systemically to a mammal a pharmaceutical composition consisting essentially of caltrin and a pharmaceutically-acceptable carrier, wherein said mammal suffers from osteopenia, and wherein said caltrin comprises an amino acid sequence selected from the group consisting of a sequence:
(a) having at least 70% homology with the residues 1-99 of SEQ ID NO: 3;
(b) having greater than 60% amino acid sequence identity with said SEQ ID NO: 3; and
wherein said caltrin induces bone formation in an in vivo bone assay.

45. A method for restoring loss of bone mass in a mammal afflicted with osteopenia, comprising administering systemically to said mammal a pharmaceutical composition consisting essentially of a caltrin and a pharmaceutically-acceptable carrier, wherein said caltrin comprises an amino acid sequence selected from the group consisting of a sequence:
(a) having at least 70% homology with the residues 1-99 of SEQ ID NO: 3;
(b) having greater than 60% amino acid sequence identity with said SEQ ID NO: 3; and
wherein said caltrin induces bone formation in an in vivo bone assay.

46. A method for preventing loss of bone mass in a mammal at risk of osteopenia, comprising administering systemically to said mammal a pharmaceutical composition consisting essentially of a caltrin and a pharmaceutically-acceptable carrier, wherein said caltrin comprises an amino acid sequence selected from the group consisting of a sequence:
(a) having at least 70% homology with the residues 1-99 of SEQ ID NO: 3;
(b) having greater than 60% amino acid sequence identity with said SEQ ID NO: 3; and
wherein said caltrin induces bone formation in an in vivo bone assay.

47. A method according to claim 44, wherein said osteopenia results from a metabolic bone disorder selected from the group consisting of osteoporosis, osteomalacia, hyperparathyroidism, Paget's disease, and renal osteodystrophy.

48. A method according to claim 44, wherein said osteopenia results from a defect in calcium or phosphate metabolism.

49. A method according to claim 44, wherein said osteopenia results from a defect in vitamin D metabolism in the mammal.

50. A method according to claim 44, wherein said osteopenia is nutritionally or hormonally induced.

51. A method according to claim 47, wherein said osteoporosis is post-menopausal or senile osteoporosis.

52. A method according to claim 17, wherein the individual has a condition selected from the group consisting of osteoporosis (including post menopausal osteoporosis, male and female senile osteoporosis and corticosteroid induced osteoporosis), osteoarthritis, Paget's disease, osteomalacia, prolonged bed rest, chronic disuse of a limb, anorexia, microgravity, exogenous and endogenous gonadal insufficiency, bone fracture, non-union, defect, prosthesis implantation, malignancy-related bone loss, and a combination of these.

53. A method according to claim 17, wherein said individual is a mammal.

54. A method according to claim 17, wherein said mammal is a human subject.

55. A method according to claim 45, wherein said osteopenia results from a metabolic bone disorder selected from the group consisting of osteoporosis, osteomalacia, hyperparathyroidism, Paget's disease, renal osteodystrophy, and a combination of these.

56. A method according to claim 45, wherein said osteopenia results from a defect in either calcium metabolism or phosphate metabolism.

57. A method according to claim 45, wherein said osteopenia results from a defect in vitamin D metabolism in the mammal.

58. A method according to claim 45, wherein said osteopenia is either nutritionally induced or hormonally induced.

59. A method according to claim 55, wherein said osteoporosis is either post-menopausal osteoporosis or senile osteoporosis.

60. A method according to claim 46, wherein said osteopenia results from a metabolic bone disorder selected from the group consisting of osteoporosis, osteomalacia, hyperparathyroidism, Paget's disease, renal osteodystrophy, and a combination of these.

61. A method according to claim 46, wherein said osteopenia results from a defect in either calcium metabolism or phosphate metabolism.

62. A method according to claim 46, wherein said osteopenia results from a defect in vitamin D metabolism in the mammal.

63. A method according to claim 46, wherein said osteopenia is either nutritionally induced or hormonally induced.

64. A method according to claim 46, wherein said osteoporosis is either post-menopausal osteoporosis or senile osteoporosis.


SEQUENCE LISTING (4 sequences)

SEQ ID NO: 1 - DNA, 500 bp, Mus musculus
atgaattcagtgacgaaaatcagcacactgctcatcgtgattttatcctttctctgttttgtggagggtctgatctgtaattcatgtgaaaagtcacgagattcaagatgtacaatgccacaaagcagatgcgttgcaaaacctggtgaatcatgcagtaccgtatcacactttgttggaacaaagcatgtgtactcaaagcaaatgtgctcgcctcagtgcaaggaaaaacagcttaacactggaaagaagttgatttacataatgtgctgcgaaaaaaacttgtgtaatagcttctagatggagaagaatcactcgaagatcatcttctgttcaagatccaaacttacaaatgaatcaagatggggaacttgctttcctactcgatttccatgatggcctgagatccctgagatagcaacagaggctgtgtcctcgtttcagtgaaagacttcttgaccacaccattagagcactgcaaaaaaaaaaaaaaaaaaaaa

SEQ ID NO: 2 - DNA, 1983 bp, Homo sapiens
aatacctcactcagcacaccgtctgtcacccaaacaagcatccaatgaggaaaatgaacacactgctccttgtgagcttatcttttctctacctcaaagaggttatgggtctgaagtgtaatacctgcatatacacagaaggatggaagtgtatggcaggccgaggcacttgcattgcaaaagaaaatgagttatgttcaacaacagcctatttcaggggagacaaacatatgtactcaacacatatgtgtaagtataagtgccgggaagaggagtcctccaaaagaggcctgttgagagtgacactgtgctgtgacagaaacttctgtaatgtcttctaatggagcttaggaacttgcagaggatcatctgatcaagatccagaatcaagaccaaccaacatgaactgttttatttcccacaccaaattccacactggcctaagatcccagagagagctgcaggggctgtcctcattgcaatgaaggggctccccacaccccacctccaccactagattcctaaaatcatgagcattgaaacaaaatcctccatgagctatgcttattcttttgtcttctactcctgatttctgatttctatcccttgtaggctaaataatggacccccaaatatttctacatcctaatctctggaacctgtgaatgttaccttacatggcaaaagggactttgaaaatgtgattaaggatcttgagaaggggaggttatcttgcattatctgggggggccctaagtggaatcaaaatgcaaaatgcaataagatgcaagtaaagggagatatgacaaagaagaggaaaagatgatgtgataatggaagcagagattggagccatgtgctttgaagatggaggaataggccataagctaaaactaggaggccactagaagctgaaaaagtcaaggaaataggttctcccctcagagcctccagaaaccagccctgctgacaccttgatttaagccctgtaagactcattttggatgtctggcctccagaactctaagcgggtgtggttttcagccacaaagtgtgtattgttttaagccactaagtttgcaataatttattatagcagcaatgggaaactaatacaatccaaataaacttctaggaattcaaatcattggtaagcctgagtacccaggggccagtctaggtgacaacagtatccaccgctcagggcttacagtgacctgcaggaagagaggaataacagagcacatgctatgaataaatgtggagatcaatttgtggattttaaacttcatgacatgctgcagataatcctttaggtgtatctgtggtaaatggtgccttgctatgttctgaagcaatcaaacatgatgtcctaatagctcaatttatcagttcctcattagcttgtgtactcactgaaattcttttagcagttaatgtccttgtattagtctgttttcatgctgctgataaggacatacccacgactgggcaatttacgaaagaaaggggtttatcggacttacagttccacgtggctggggaagcctcacaatcatggcagaaggtaaaaggcatgtctcacatagtgacagacaagagaagagagattgtgcaggaaaactctcccttataataaccatcagatcttgtgagacttactcactatcacgagaatagcacaggaaaaacctgcccccatgattcaattacctcccaccggtccctcccacaacacgtgggaattgaagatgaaatttgggtggggacacagccaaaccatatcagtccccttcttaaaactctcctctctagctcctatgactgtacatttgtagttctcccacctctctgaagaatcctctgtgagggtctctggggactttgttttgatttgttattgtttttatattcagtctttaacctatgtgaagccacctttgtaatactgccttaagtaaagatccagtattatttttctcc

SEQ ID NO: 3 - protein, 99 aa, Mus musculus
MNSVTKISTLLIVILSFLCFVEGLICNSCEKSRDSRCTMPQSRCVAKPGESCSTVSHFVGTKHVYSKQMCSPQCKEKQLNTGKKLIYIMCCEKNLCNSF

SEQ ID NO: 4 - protein, 96 aa, Homo sapiens
MRKMNTLLLVSLSFLYLKEGLKCNTCIYTEGWKCMAGRGTCIAKENELCSTTAYFSGDKHMYSTHMCKYKCREEESSKRGLLRVTLCCDRNFCNVF